(19) United States
(12) Patent Application Publication
(10) Pub. No.: US 2026/0167707 A1
(43) Pub. Date: Jun. 18, 2026

(54) IL-13 ANTIBODIES FOR THE TREATMENT OF ATOPIC DERMATITIS

(71) Applicant: DERMIRA, INC., Indianapolis, IN (US)

(72) Inventors: Eugene A. BAUER, San Rafael, CA (US); Janice Suzann DREW, Indianapolis, IN (US); Renata Gontijo LIMA, Ponte Vedra, FL (US); Jennifer Wright WITCHER, Atlanta, GA (US)

(21) Appl. No.: 18/682,575

(22) PCT Filed: Aug. 12, 2022
(86) PCT No.: PCT/US2022/074926

§ 371 Date: Feb. 9, 2024

Related U.S. Application Data
(60) Provisional application No. 63/232,977, filed on Aug. 13, 2021.
(60) Provisional application No. 63/233,544, filed on Aug. 16, 2021.
(60) Provisional application No. 63/247,821, filed on Sep. 24, 2021.
(60) Provisional application No. 63/327,539, filed on Apr. 5, 2022.
(60) Provisional application No. 63/343,315, filed on May 18, 2022.

(51) Int. Cl. C07K 16/24; A61K 39/00; A61K 45/06; A61P 17/00

(52) U.S. Cl. CPC C07K 16/244; A61K 45/06; A61P 17/00; A61K 2039/505; A61K 2039/54; A61K 2039/545; C07K 2317/565

(57) ABSTRACT

Provided herein are methods, uses, and pharmaceutical compositions of antibodies that bind human IL-13 (“anti-IL-13 antibodies”) for treating atopic dermatitis. Also provided herein are dosing regimens for the methods and uses of anti-IL-13 antibodies for treating atopic dermatitis.

### SEQUENCE LISTING

The present application is being filed along with a Sequence Listing in ST.26 XML format. The Sequence Listing is provided as a file titled “X23063_SequenceListing” created 3-August-2022 and is 15 kilobytes in size. The Sequence Listing information in the ST.26 XML format is incorporated herein by reference in its entirety.

### FIELD

The present invention relates to methods, uses, and pharmaceutical compositions of antibodies that bind human IL-13 (“anti-IL-13 antibodies”) for treating atopic dermatitis. The present invention also relates to dosing regimens for the methods and uses of anti-IL-13 antibodies for treating atopic dermatitis.

### BACKGROUND

Atopic dermatitis (AD) is a chronic relapsing and remitting inflammatory skin disorder affecting all age groups. Clinically, AD is characterized by xerosis, erythematous crusting rash, lichenification, an impaired skin barrier, and intense pruritus (Bieber T., N Engl J Med 2008; 358:1483-94). Patients with AD have a high disease burden, and their quality of life is significantly impacted. In one study, AD was shown to have a greater negative effect on patient mental health than diabetes and hypertension (Zuberbier T, et al., J Allergy Clin Immunol 2006; 118:226-32). Patients with moderate to severe AD have a higher prevalence of social dysfunction and sleep impairment, which is directly related to severity of disease (Williams H, et al., J Allergy Clin Immunol 2008; 121:947-54.e15). Depression, anxiety, and social dysfunction not only affect patients with AD but also their caregivers (Zuberbier T, et al., J Allergy Clin Immunol 2006; 118:226-32).

Interleukin (IL)-13 is a key mediator of T-helper type 2 (Th2) inflammation and signals through a heterodimeric receptor IL-4Rα/IL-13Rα1. Several lines of evidence suggest that IL-13 is a key pathogenetic component in AD. Increased expression of IL-13 has consistently been reported in AD skin (Hamid Q, et al., J Allergy Clin Immunol 98:225-31 [1996]; Jeong C W, et al., Clin Exp Allergy 33:1717-24 [2003]; Tazawa T, et al., Arch Dermatol Res 295:459-64 [2004]; Neis M M, et al., J Allergy Clin Immunol 118:930-7 [2006]; Suárez-Fariñas M, et al., J Allergy Clin Immunol 132:361-70 [2013]; Choy D F, et al., J Allergy Clin Immunol. 130:1335-43 [2012]) and some reports suggest a relationship between IL-13 expression and the severity of disease (La Grutta S, et al., Allergy 60:391-5 [2005]). Increased IL-13 has also been reported in the serum of AD patients (Novak N, et al., J Invest Dermatol 2002; 119:870-5; WO2016149276), and several studies have reported an increase in IL-13-expressing T cells in the blood of AD patients (Akdis M, et al., J Immunol 1997; 159:4611-9; Aleksza M, et al., Br J Dermatol 2002; 147:1135-41; La Grutta S, et al., Allergy 2005; 60:391-5).

The therapeutic approaches to AD primarily include trigger avoidance, skin hydration with bathing and use of emollients and anti-inflammatory therapies such as topical corticosteroids (TCS). In many patients, treatment with TCS provides some measure of symptomatic relief but does not adequately control their disease. In addition, TCS use is associated with many comorbidities and limitations including high patient burden. Long-term application of TCS is not recommended because of the risk of skin atrophy, dyspigmentation, acneiform eruptions, and risks associated with systemic absorption (e.g., hypothalamic pituitary axis effects, Cushing's disease). Topical calcineurin inhibitors (TCI) are generally effective and safe as short-term treatments, but concerns of skin malignancies and increased risk of lymphomas have prompted regulatory authorities to require a warning regarding the long-term safety of topical tacrolimus and pimecrolimus in their prescribing information. Repeated application of any topical therapy over a long period of time or to large surface areas also leads to reduced patient compliance.

For patients who have persistent moderate to severe AD and do not respond adequately to TCS, there are a number of step-up therapeutic options (Ring J, et al., J Eur Acad Dermatol Venereol 2012; 26:1176-93; Schneider L, et. al., J Allergy Clin Immunol 2013; 131:295-9. e1-27). Oral immunosuppressants (Schmitt et al 2007, JEADV 21:606-619) and glucocorticoids are effective, but are sometimes associated with severe toxicity and side effects, thus limiting their use to short courses and/or intermittent therapy. Cyclosporine is approved for treatment of moderate to severe AD in many European countries, but not in the United States, and its use is limited to patients aged 16 years and over (for a maximum of 8 weeks [NEORAL®]). Even in cases where cyclosporine has demonstrated substantial efficacy, approximately 50% of patients relapse within 2 weeks, and 80% relapse within 6 weeks after cessation of therapy (Amor K T, et al., J Am Acad Dermatol 2010; 63:925-46). Cyclosporine A (CsA) is a potent immunosuppressant affecting both humoral and cellular immune responses, which could lead to increased susceptibility to infections and decreased cancer immunosurveillance. Other commonly recognized toxicities of CsA include hypertension and impaired renal and hepatic function. In addition, CsA interacts with other commonly used medicines potentially affecting their metabolism and effect.

There remains an unmet medical need for safer and more effective therapies and treatment regimens for moderate to severe AD. There is also a need for therapeutic treatments and dosing regimens that provide higher tolerability and convenience and lower risk for patients, thereby improving patient compliance and satisfaction.

### SUMMARY OF INVENTION

Provided herein are methods, uses, and pharmaceutical compositions of anti-IL-13 antibodies (e.g., lebrikizumab) for treating atopic dermatitis. Also provided herein are dosing regimens for the methods and uses of anti-IL-13 antibodies (e.g., lebrikizumab) for treating atopic dermatitis. The methods and dosing regimens provided herein have one or more of the following advantages: optimized and improved dosing frequency that enables higher patient compliance and higher patient satisfaction while maintaining the desired efficacy; lower risk of injection site reactions; and/or lower manufacturing costs.

In one aspect, provided herein are methods of treating moderate to severe atopic dermatitis in a patient in need thereof, the method comprising: administering to the patient an anti-IL-13 antibody for an induction period (or a first period) of up to 16 weeks (e.g., 4 to 16 weeks), wherein, during the induction period (or the first period), the anti-IL-13 antibody is administered at 500 mg at baseline (week 0) and week 2, followed by 250 mg once every two weeks for 2 to 14 weeks; and administering to the patient the anti-IL-13 antibody 250 mg once every four weeks for a maintenance period (or a second period) of up to 36 weeks (e.g., 8 to 36 weeks). In some embodiments, the induction period (or the first period) is 16 weeks. During the induction period (or the first period), the anti-IL-13 antibody is administered at 500 mg at baseline (week 0) and week 2, followed by 250 mg once every two weeks for 14 weeks. In some embodiments, the maintenance period (or the second period) is 36 weeks. In some embodiments, the anti-IL-13 antibody is administered to the patient subcutaneously.

In some embodiments, provided herein are methods of treating moderate to severe atopic dermatitis in a patient in need thereof, and such methods comprise administering to the patient an anti-IL-13 antibody for an induction period (or a first period) of up to 16 weeks (e.g., 4 to 16 weeks), wherein, during the induction period (or the first period), the anti-IL-13 antibody is administered at 500 mg at baseline (week 0) and week 2, followed by 250 mg once every two weeks for 2 to 14 weeks; and administering to the patient the anti-IL-13 antibody 250 mg once every two weeks for a maintenance period (or a second period) of up to 36 weeks (e.g., 8 to 36 weeks). In some embodiments, the induction period (or the first period) is 16 weeks. During the induction period (or the first period), the anti-IL-13 antibody is administered at 500 mg at baseline (week 0) and week 2, followed by 250 mg once every two weeks for 14 weeks. In some embodiments, the maintenance period (or the second period) is 36 weeks. In some embodiments, the anti-IL-13 antibody is administered to the patient subcutaneously.

Also provided herein are methods of treating moderate to severe atopic dermatitis in a patient in need thereof, the method comprising: administering to the patient an anti-IL-13 antibody for an induction period (or a first period) of up to 16 weeks (e.g., 4 to 16 weeks), wherein, during the induction period (or the first period), the anti-IL-13 antibody is administered at 500 mg at baseline (week 0) and week 2, followed by 250 mg once every two weeks for 2 to 14 weeks; determining if the patient is a responder to the anti-IL-13 antibody after the induction period (or the first period); and if the patient is a responder, administering to the patient the anti-IL-13 antibody at 250 mg once every two weeks or once every four weeks or once every eight weeks for a maintenance period (or a second period) of up to 36 weeks (e.g., 8 to 36 weeks); if the patient is not a responder, administering to the patient the anti-IL-13 antibody at 250 mg once every two weeks for a maintenance period (or a second period) of up to 36 weeks (e.g., 8 to 36 weeks). In some embodiments, the induction period (or the first period) is 16 weeks. During the induction period (or the first period), the anti-IL-13 antibody is administered at 500 mg at baseline (week 0) and week 2, followed by 250 mg once every two weeks for 14 weeks. In some embodiments, the maintenance period (or the second period) is 36 weeks. Determining if the patient is a responder to the anti-IL-13 antibody can be assessed by evaluating the patient's skin clearance, skin improvement, and/or improvement in itch, sleep, or quality of life. For example, skin clearance and skin improvement can be measured by Investigator Global Assessment (IGA) or Eczema Area and Severity Index (EASI) scores. Itch, sleep loss and quality of life can be measured by Pruritus Numerical Rating Scale (NRS), Sleep Loss score and DLQI (Dermatology Life Quality Index) or CDLQI (Children Dermatology Life Quality Index) scales, respectively. In some embodiments, the patient is a responder when the patient's EASI score determined after the induction period (or the first period) is reduced by 75% or greater compared to the patient's EASI score at the baseline. In some embodiments, the patient is a responder when the patient's IGA score is 0 or 1 after the induction period (or the first period). In some embodiments, the patient is a responder when the patient's IGA score is 0 or 1 after the induction period (or the first period) and the patient's IGA score determined after the induction period (or the first period) is reduced by 2 points or greater compared to the patient's IGA score at the baseline. In some embodiments, if the patient is a responder, the anti-IL-13 antibody is administered at 250 mg once every four weeks for the maintenance period (or the second period). In some embodiments, the anti-IL-13 antibody is administered to the patient subcutaneously.

In some embodiments, provided herein are methods of treating moderate to severe atopic dermatitis in a patient in need thereof, the method comprising: administering to the patient an anti-IL-13 antibody for a first period of 4 to 16 weeks, wherein, during the first period, the anti-IL-13 antibody is administered at 500 mg at baseline (week 0) and week 2, followed by 250 mg once every two weeks for 2 to 14 weeks; and administering to the patient the anti-IL-13 antibody 250 mg once every four weeks or once every eight weeks for a second period of 8 to 36 weeks, wherein the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

In some embodiments, provided herein are methods of treating moderate to severe atopic dermatitis in a patient in need thereof, the method comprising: administering to the patient an anti-IL-13 antibody for a first period of 4 to 16 weeks, wherein, during the first period, the anti-IL-13 antibody is administered at 500 mg at baseline (week 0) and week 2, followed by 250 mg once every two weeks for 2 to 14 weeks; determining if the patient is a responder to the anti-IL-13 antibody after the first period; and if the patient is a responder, administering to the patient the anti-IL-13 antibody at 250 mg once every four weeks or once every eight weeks for a second period of 8 to 36 weeks; if the patient is not a responder, administering to the patient the anti-IL-13 antibody at 250 mg once every two weeks for a second period of 8 to 36 weeks, wherein the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

In some embodiments, the patient has moderate to severe atopic dermatitis for at least a year at the baseline. The moderate to severe atopic dermatitis can be determined by criteria known in the art, e.g., the American Academy of Dermatology Consensus Criteria for Chronic Atopic Dermatitis. In some embodiments, the patient has an EASI score of 16 or greater, an IGA score of 3 or greater, and more than 10% of body surface area (BSA) affected by atopic dermatitis, at the baseline. In some embodiments, the patient has inadequate response to topical corticosteroids, topical calcineurin inhibitors, or crisaborole; or topical corticosteroids, topical calcineurin inhibitors, or crisaborole are medically inadvisable for the patient. In some embodiments, the patient is aged 12 years and older.

In some embodiments, the methods described herein further comprise determining one or more of the following characteristics of the patient at baseline and during and after the induction period (or the first period): the EASI score; the IGA score; the percentage of BSA affected by atopic dermatitis; pruritus NRS score; SCORAD (Scoring of Atopic Dermatitis) score; sleep loss score; POEM (Patient-Oriented Eczema Measure) total score; DLQI (Dermatology Life Quality Index) or CDLQI (Children Dermatology Life Quality Index) score; EQ-5D (European Quality of Life-5 Dimensions); ACQ-5 (Asthma Control Questionnaire-5); PROMIS (Patient-Reported Outcomes Measurement Information System) Anxiety and Depressive Symptoms.

In some embodiments, the methods described herein further comprise determining one or more of the following characteristics of the patient during and after the maintenance period (or the second period): the EASI score; the IGA score; the percentage of BSA affected by atopic dermatitis; pruritus NRS score; SCORAD score; sleep loss score; POEM total score; DLQI or CDLQI score; EQ-5D; ACQ-5; PROMIS Anxiety and Depressive Symptoms.

In another aspect, provided herein are methods of reducing sleep loss in a patient with moderate to severe atopic dermatitis; such methods comprise administering to the patient an anti-IL-13 antibody (e.g., lebrikizumab) at a loading dose of 500 mg at baseline (week 0) and week 2, followed by a subsequent dose of 250 mg once every two weeks. In some embodiments, the anti-IL-13 antibody is administered to the patient for a period of 4 to 52 weeks. In some embodiments, the anti-IL-13 antibody is administered to the patient for a period of 4 to 16 weeks. In some embodiments, the sleep loss is determined by the patient's sleep loss score. In some embodiments, the patient's sleep loss score after the anti-IL-13 antibody treatment is reduced for two points or greater compared to the patient's sleep score at baseline.

Also provided herein are methods of reducing sleep loss in a patient with moderate to severe atopic dermatitis; such methods comprise administering to the patient an anti-IL-13 antibody (e.g., lebrikizumab) for an induction period (or a first period) of 4 to 16 weeks, wherein, during the induction period (or the first period), the anti-IL-13 antibody is administered at 500 mg at baseline (week 0) and week 2, followed by 250 mg once every two weeks for 2 to 14 weeks; and administering to the patient the anti-IL-13 antibody 250 mg once every two weeks or once every four weeks or once every eight weeks for a maintenance period (or a second period) of 8 to 36 weeks. In some embodiments, the sleep loss is determined by the patient's sleep loss score. In some embodiments, the patient's sleep loss score after the anti-IL-13 antibody treatment is reduced for two points or greater compared to the patient's sleep score at baseline.

Also provided herein are methods of reducing sleep loss in a patient with moderate to severe atopic dermatitis; such methods comprise administering to the patient an anti-IL-13 antibody (e.g., lebrikizumab) for an induction period (or a first period) of 4 to 16 weeks, wherein, during the induction period (or the first period), the anti-IL-13 antibody is administered at 500 mg at baseline (week 0) and week 2, followed by 250 mg once every two weeks for 2 to 14 weeks; determining if the patient is a responder to the anti-IL-13 antibody after the induction period (or the first period); and if the patient is a responder, administering to the patient the anti-IL-13 antibody at 250 mg once every two weeks or once every four weeks or once every eight weeks for a maintenance period (or a second period) of 8 to 36 weeks; if the patient is not a responder, administering to the patient the anti-IL-13 antibody at 250 mg once every two weeks for a maintenance period (or a second period) of 8 to 36 weeks. In some embodiments, the sleep loss is determined by the patient's sleep loss score. In some embodiments, the patient's sleep loss score after the anti-IL-13 antibody treatment is reduced for two points or greater compared to the patient's sleep score at baseline.

In some embodiments, the methods described herein further comprise determining one or more of the following characteristics of the patient: the EASI score; the IGA score; the percentage of BSA affected by atopic dermatitis; pruritus NRS score; SCORAD score; sleep loss score; POEM total score; DLQI or CDLQI score; EQ-5D; ACQ-5; PROMIS Anxiety and Depressive Symptoms.

In some embodiments, the anti-IL-13 antibody binds IL-13 with high affinity and blocks signaling through the active IL-4Ralpha/IL-13Ralpha1 heterodimer. In some embodiments, the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6. In some embodiments, the anti-IL-13 antibody comprises a VH comprising SEQ ID NO: 7, and a VL comprising SEQ ID NO: 8. In some embodiments, the anti-IL-13 antibody comprises a heavy chain comprising SEQ ID NO: 9, and a light chain comprising SEQ ID NO: 10. In some embodiments, the anti-IL-13 antibody is lebrikizumab.

In another aspect, provided herein are anti-IL-13 antibody or pharmaceutical composition comprising an anti-IL-13 antibody for use in the treatment of moderate to severe atopic dermatitis in a patient.

In another aspect, provided herein are anti-IL-13 antibody or a pharmaceutical composition comprising an anti-IL-13 antibody, for use in a method of reducing sleep loss in a patient with moderate to severe atopic dermatitis. In another aspect, provided herein are anti-IL-13 antibody or a pharmaceutical composition comprising an anti-IL-13 antibody, for use in reducing sleep loss in a patient with moderate to severe atopic dermatitis.

In some embodiments, provided herein are anti-IL-13 antibody or a pharmaceutical composition comprising an anti-IL-13 antibody, for use in reducing sleep loss in a patient with moderate to severe atopic dermatitis, wherein the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6, and wherein the anti-IL-13 antibody or pharmaceutical composition is for administration at a loading dose of 500 mg at baseline (week 0) and week 2, followed by a subsequent dose of 250 mg once every two weeks.

In some embodiments, provided herein are anti-IL-13 antibody or a pharmaceutical composition comprising an anti-IL-13 antibody, for use in reducing sleep loss in a patient with moderate to severe atopic dermatitis, wherein the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6, and wherein the anti-IL-13 antibody or pharmaceutical composition is for administration for an induction period (or a first period) of 4 to 16 weeks, and during the induction period (or the first period), the anti-IL-13 antibody is for administration at 500 mg at baseline (week 0) and week 2, followed by 250 mg once every two weeks for 2 to 14 weeks; and wherein the anti-IL-13 antibody is for administration at 250 mg once every four weeks for a maintenance period (or a second period) of 8 to 36 weeks.

In another aspect, provided herein are uses of an anti-IL-13 antibody in the manufacture of a medicament for the treatment of moderate to severe atopic dermatitis in a patient. Also provided herein are uses of an anti-IL-13 antibody in the manufacture of a medicament for reducing sleep loss in a patient with moderate to severe atopic dermatitis.

In some embodiments, the methods, uses, and pharmaceutical compositions described herein further comprise administrating one or more topical corticosteroids to the patient. In some embodiments, the topical corticosteroid is triamcinolone acetonide, hydrocortisone, or a combination of triamcinolone acetonide and hydrocortisone. In some embodiments, the topical corticosteroids are administered concomitantly with the anti-IL-13 antibody.

### BRIEF DESCRIPTION OF THE DRAWINGS

FIG. 1 is a schematic diagram of the Phase 3 study designs described in Example 1.

FIGS. 2A and 2B show baseline demographics of the participants in ADvocate 1; and FIG. 2C shows baseline disease characteristics of the participants in ADvocate 1. AD=Atopic Dermatitis; ITT=Intent-to-Treat; LEB=Lebrikizumab; Q2W=Every 2 Weeks; PBO=Placebo; SD=Standard Deviation; BMI=Body-Mass Index; BSA=Body Surface Area; DLQI-Dermatology Life Quality Index; EASI=Eczema Area and Severity Index; IGA-Investigator's Global Assessment; IQR=Interquartile Range; NRS-Numerical Rating Scale; POEM=Patient-Oriented Eczema Measure; SCORAD=Scoring Atopic Dermatitis.

FIG. 3A shows the overview of adverse events through Week 16 in ADvocate 1. FIG. 3B shows the serious adverse events through Week 16 in ADvocate 1. FIG. 3C shows the TEAEs within special safety topics through Week 16 in ADvocate 1. FIG. 3D shows the injection site reactions through Week 16 in ADvocate 1. AE=Adverse Event; LEB=Lebrikizumab; Q2W=Every 2 Weeks; PBO=Placebo; TEAE=Treatment-Emergent Adverse Event.

FIG. 4A shows the overview of achievement of key efficacy endpoints in ADvocate 1. FIG. 4B shows the IGA response rates at Week 16 in ADvocate 1, which measures the percentage of IGA (0,1) with ≥2-point improvement from baseline at Week 16. FIG. 4C shows the EASI-75 response rates at Week 16 in ADvocate 1. FIG. 4D shows the IGA response rates over time up to Week 16 in ADvocate 1. FIG. 4E shows the EASI-75 response rates over time up to Week 16 in ADvocate 1. FIG. 4F shows the EASI-90 response rates over time up to Week 16 in ADvocate 1. FIG. 4G shows EASI percent change from baseline over time up to Week 16 in ADvocate 1.

FIG. 5A shows the pruritus NRS≥4-point improvement from baseline over time up to Week 16 in ADvocate 1. FIG. 5B shows pruritus NRS percent change from baseline over time up to Week 16 in ADvocate 1. FIG. 5C shows the sleep-loss score ≥2-point improvement from baseline over time up to Week 16 in ADvocate 1. FIG. 5D shows sleep-loss score change from baseline over time up to Week 16 in ADvocate 1. FIG. 5E shows DLQI≥4-point improvement from baseline over time up to Week 16. FIG. 5F shows DLQI change from baseline over time up to Week 16 in ADvocate 1.

FIGS. 6A and 6B show the IGA response rates at Week 52 in ADvocate 1 (6A) and ADvocate 2 (6B), which measure the percentage of patients who achieved IGA (0,1). FIGS. 6C and 6D show the EASI-75 response rates at Week 52 in ADvocate 1 (6C) and ADvocate 2 (6D), which measure the percentage of patients who achieved EASI-75. FIGS. 6E and 6F show itch response rates at Week 52 in ADvocate 1 (6E) and ADvocate 2 (6F), which measure the percentage of patients who had Pruritus NRS≥4 at baseline and achieved ≥4-point improvement.

FIG. 7 shows the overview of adverse events from Week 16 to Week 52 in ADvocate 1 and ADvocate 2.

FIG. 8A is a graphical illustration of the final PK-PD model in Example 2. FIG. 8B shows the model parameter estimates of the final PK-PD model.

FIG. 9 shows the simulated EASI-75 response rate for responders at Week 16 who went to various dosing maintenance regimens for Week 16 to 52. Line shows the median across 500 simulations.

FIG. 10 shows the simulated EASI-75 response rate for responders at Week 16 who went to lebrikizumab 250 mg Q4W or 250 mg Q8W maintenance regimens for Week 16 to 52. Line shows the median across 500 simulations and shaded region shows the 95% confidence interval.

FIG. 11 shows the simulated EASI-90 response rate for responders at Week 16 who went to various dosing regimens for Week 16 to 52. Line shows the median across 500 simulations.

FIG. 12 shows the simulated EASI-90 response rate for responders at Week 16 who went to lebrikizumab 250 mg Q4W or 250 mg Q8W maintenance regimens for Week 16 to 52. Line shows the median across 500 simulations and shaded region shows the 95% confidence interval.

### DETAILED DESCRIPTION

Provided herein are methods, uses, and pharmaceutical compositions of anti-IL-13 antibodies for treating atopic dermatitis. Also provided herein are dosing regimens for the methods and uses of anti-IL-13 antibodies for treating atopic dermatitis. The methods and dosing regimens provided herein have one or more of the following advantages: optimized and/or improved dosing frequency that enables higher patient compliance and higher patient satisfaction while maintaining the desired efficacy; lower risk of injection site reactions; lower manufacturing costs.

In one aspect, provided herein are methods of treating moderate to severe atopic dermatitis in a patient in need thereof, the method comprising: administering to the patient an anti-IL-13 antibody for an induction period (or a first period) of up to 16 weeks (e.g., 4 to 16 weeks), wherein, during the induction period (or the first period), the anti-IL-13 antibody is administered at 500 mg at baseline (week 0) and week 2, followed by 250 mg once every two weeks for 2 to 14 weeks; and administering to the patient the anti-IL-13 antibody 250 mg once every four weeks for a maintenance period (or a second period) of up to 36 weeks (e.g., 8 to 36 weeks). In some embodiments, the induction period (or the first period) is 16 weeks. During the 16 week induction period (or first period), the anti-IL-13 antibody is administered at 500 mg at baseline (week 0) and week 2, followed by 250 mg once every two weeks for 14 weeks. In some embodiments, the maintenance period (or the second period) is 36 weeks. In some embodiments, the anti-IL-13 antibody is administered to the patient subcutaneously.

In one aspect, provided herein are methods of treating moderate to severe atopic dermatitis in a patient in need thereof, the method comprising: administering to the patient an anti-IL-13 antibody for an induction period (or a first period) of up to 16 weeks (e.g., 4 to 16 weeks), wherein, during the induction period (or the first period), the anti-IL-13 antibody is administered at 500 mg at baseline (week 0) and week 2, followed by 250 mg once every two weeks for 2 to 14 weeks; and administering to the patient the anti-IL-13 antibody 250 mg once every eight weeks for a maintenance period (or a second period) of up to 36 weeks (e.g., 8 to 36 weeks). In some embodiments, the induction period (or the first period) is 16 weeks. During the 16 week induction period (or first period), the anti-IL-13 antibody is administered at 500 mg at baseline (week 0) and week 2, followed by 250 mg once every two weeks for 14 weeks. In some embodiments, the maintenance period (or the second period) is 36 weeks. In some embodiments, the anti-IL-13 antibody is administered to the patient subcutaneously.

In some embodiments, provided herein are methods of treating moderate to severe atopic dermatitis in a patient in need thereof, and such methods comprise administering to the patient an anti-IL-13 antibody for an induction period (or a first period) of up to 16 weeks (e.g., 4 to 16 weeks), wherein, during the induction period (or the first period), the anti-IL-13 antibody is administered at 500 mg at baseline (week 0) and week 2, followed by 250 mg once every two weeks for 2 to 14 weeks; and administering to the patient the anti-IL-13 antibody 250 mg once every two weeks for a maintenance period (or a second period) of up to 36 weeks (e.g., 8 to 36 weeks). In some embodiments, the induction period (or the first period) is 16 weeks; and during the induction period (or the first period), the anti-IL-13 antibody is administered at 500 mg at baseline (week 0) and week 2, followed by 250 mg once every two weeks for 14 weeks. In some embodiments, the maintenance period (or the second period) is 36 weeks. In some embodiments, the anti-IL-13 antibody is administered to the patient subcutaneously.

Also provided herein are methods of treating moderate to severe atopic dermatitis in a patient in need thereof, the method comprising: administering to the patient an anti-IL-13 antibody for an induction period (or a first period) of up to 16 weeks (e.g., 4 to 16 weeks), wherein, during the induction period (or the first period), the anti-IL-13 antibody is administered at 500 mg at baseline (week 0) and week 2, followed by 250 mg once every two weeks for 2 to 14 weeks; determining if the patient is a responder to the anti-IL-13 antibody after the induction period (or the first period); and if the patient is a responder, administering to the patient the anti-IL-13 antibody at 250 mg once every two weeks or once every four weeks for a maintenance period (or a second period) of up to 36 weeks (e.g., 8 to 36 weeks); if the patient is not a responder, administering to the patient the anti-IL-13 antibody at 250 mg once every two weeks for a maintenance period (or a second period) of up to 36 weeks (e.g., 8 to 36 weeks). In some embodiments, the induction period (or the first period) is 16 weeks. During the induction period (or the first period), the anti-IL-13 antibody is administered at 500 mg at baseline (week 0) and week 2, followed by 250 mg once every two weeks for 14 weeks. In some embodiments, the maintenance period (or the second period) is 36 weeks. In some embodiments, the anti-IL-13 antibody is administered to the patient subcutaneously.

Also provided herein are methods of treating moderate to severe atopic dermatitis in a patient in need thereof, the method comprising: administering to the patient an anti-IL-13 antibody for an induction period (or a first period) of up to 16 weeks (e.g., 4 to 16 weeks), wherein, during the induction period (or the first period), the anti-IL-13 antibody is administered at 500 mg at baseline (week 0) and week 2, followed by 250 mg once every two weeks for 2 to 14 weeks; determining if the patient is a responder to the anti-IL-13 antibody after the induction period (or the first period); and if the patient is a responder, administering to the patient the anti-IL-13 antibody at 250 mg once every two weeks or once every eight weeks for a maintenance period (or a second period) of up to 36 weeks (e.g., 8 to 36 weeks); if the patient is not a responder, administering to the patient the anti-IL-13 antibody at 250 mg once every two weeks for a maintenance period (or a second period) of up to 36 weeks (e.g., 8 to 36 weeks). In some embodiments, the induction period (or the first period) is 16 weeks. During the induction period (or the first period), the anti-IL-13 antibody is administered at 500 mg at baseline (week 0) and week 2, followed by 250 mg once every two weeks for 14 weeks. In some embodiments, the maintenance period (or the second period) is 36 weeks. In some embodiments, the anti-IL-13 antibody is administered to the patient subcutaneously.

Determining if the patient is a responder to the anti-IL-13 antibody can be assessed by evaluating the patient's skin clearance, skin improvement, and/or improvement in itch, sleep, or quality of life. For example, skin clearance and skin improvement can be measured by IGA or EASI scores. Itch, sleep loss and quality of life can be measured by pruritus NRS, Sleep Loss score and DLQI or CDLQI scales, respectively. In some embodiments, the patient is a responder when the patient's EASI score determined after the induction period (or the first period) is reduced by 75% or greater compared to the patient's EASI score at the baseline. In some embodiments, the patient is a responder when the patient's IGA score is 0 or 1 after the induction period (or the first period). In some embodiments, the patient is a responder when the patient's IGA score is 0 or 1 after the induction period (or the first period) and the patient's IGA score determined after the induction period (or the first period) is reduced by 2 points or greater compared to the patient's IGA score at the baseline. In some embodiments, if the patient is a responder, the anti-IL-13 antibody is administered at 250 mg once every four weeks for the maintenance period (or the second period). In some embodiments, if the patient is a responder, the anti-IL-13 antibody is administered at 250 mg once every eight weeks for the maintenance period (or the second period).

In another aspect, provided herein are methods of reducing sleep loss in a patient with moderate to severe atopic dermatitis; such methods comprise administering to the patient an anti-IL-13 antibody (e.g., lebrikizumab) at a loading dose of 500 mg at baseline (week 0) and week 2, followed by a subsequent dose of 250 mg once every two weeks. In some embodiments, the anti-IL-13 antibody is administered to the patient for a period of 4 to 52 weeks (e.g., about 4 weeks, about 6 weeks, about 8 weeks, about 10 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks). In some embodiments, the anti-IL-13 antibody is administered to the patient for a period of 4 to 16 weeks (e.g., about 4 weeks, about 6 weeks, about 8 weeks, about 10 weeks, about 12 weeks, about 14 weeks, about 16 weeks). In some embodiments, the sleep loss is determined by the patient's sleep loss score, e.g., as described herein. In some embodiments, the patient's sleep loss score after the anti-IL-13 antibody treatment is reduced for two points or greater compared to the patient's sleep score at baseline.

Also provided herein are methods of reducing sleep loss in a patient with moderate to severe atopic dermatitis; such methods comprise administering to the patient an anti-IL-13 antibody (e.g., lebrikizumab) for an induction period (or a first period) of 4 to 16 weeks, wherein, during the induction period (or the first period), the anti-IL-13 antibody is administered at 500 mg at baseline (week 0) and week 2, followed by 250 mg once every two weeks for 2 to 14 weeks; and administering to the patient the anti-IL-13 antibody 250 mg once every two weeks or once every four weeks for a maintenance period (or a second period) of 8 to 36 weeks. In some embodiments, the sleep loss is determined by the patient's sleep loss score. In some embodiments, the patient's sleep loss score after the anti-IL-13 antibody treatment is reduced for two points or greater compared to the patient's sleep score at baseline. In some embodiments, the induction period (or the first period) is 16 weeks, and during the induction period (or the first period), the anti-IL-13 antibody is administered at 500 mg at baseline (week 0) and week 2, followed by 250 mg once every two weeks for 14 weeks. In some embodiments, the maintenance period (or the second period) is 36 weeks. In some embodiments, the anti-IL-13 antibody is administered to the patient subcutaneously.

Also provided herein are methods of reducing sleep loss in a patient with moderate to severe atopic dermatitis; such methods comprise administering to the patient an anti-IL-13 antibody (e.g., lebrikizumab) for an induction period (or a first period) of 4 to 16 weeks, wherein, during the induction period (or the first period), the anti-IL-13 antibody is administered at 500 mg at baseline (week 0) and week 2, followed by 250 mg once every two weeks for 2 to 14 weeks; and administering to the patient the anti-IL-13 antibody 250 mg once every eight weeks for a maintenance period (or a second period) of 8 to 36 weeks. In some embodiments, the sleep loss is determined by the patient's sleep loss score. In some embodiments, the patient's sleep loss score after the anti-IL-13 antibody treatment is reduced for two points or greater compared to the patient's sleep score at baseline. In some embodiments, the induction period (or the first period) is 16 weeks, and during the induction period (or the first period), the anti-IL-13 antibody is administered at 500 mg at baseline (week 0) and week 2, followed by 250 mg once every two weeks for 14 weeks. In some embodiments, the maintenance period (or the second period) is 36 weeks. In some embodiments, the anti-IL-13 antibody is administered to the patient subcutaneously.

Also provided herein are methods of reducing sleep loss in a patient with moderate to severe atopic dermatitis; such methods comprise administering to the patient an anti-IL-13 antibody for an induction period (or a first period) of 4 to 16 weeks, wherein, during the induction period (or the first period), the anti-IL-13 antibody is administered at 500 mg at baseline (week 0) and week 2, followed by 250 mg once every two weeks for 2 to 14 weeks; determining if the patient is a responder to the anti-IL-13 antibody after the induction period (or the first period); and if the patient is a responder, administering to the patient the anti-IL-13 antibody at 250 mg once every two weeks or once every four weeks for a maintenance period (or a second period) of 8 to 36 weeks; if the patient is not a responder, administering to the patient the anti-IL-13 antibody at 250 mg once every two weeks for a maintenance period (or a second period) of 8 to 36 weeks. In some embodiments, the sleep loss is determined by the patient's sleep loss score. In some embodiments, the patient's sleep loss score after the anti-IL-13 antibody treatment is reduced for two points or greater compared to the patient's sleep score at baseline. In some embodiments, the induction period (or the first period) is 16 weeks, and during the induction period (or the first period), the anti-IL-13 antibody is administered at 500 mg at baseline (week 0) and week 2, followed by 250 mg once every two weeks for 14 weeks. In some embodiments, the maintenance period (or the second period) is 36 weeks. In some embodiments, the anti-IL-13 antibody is administered to the patient subcutaneously.

Also provided herein are methods of reducing sleep loss in a patient with moderate to severe atopic dermatitis; such methods comprise administering to the patient an anti-IL-13 antibody for an induction period (or a first period) of 4 to 16 weeks, wherein, during the induction period (or the first period), the anti-IL-13 antibody is administered at 500 mg at baseline (week 0) and week 2, followed by 250 mg once every two weeks for 2 to 14 weeks; determining if the patient is a responder to the anti-IL-13 antibody after the induction period (or the first period); and if the patient is a responder, administering to the patient the anti-IL-13 antibody at 250 mg once every eight weeks for a maintenance period (or a second period) of 8 to 36 weeks; if the patient is not a responder, administering to the patient the anti-IL-13 antibody at 250 mg once every two weeks for a maintenance period (or a second period) of 8 to 36 weeks. In some embodiments, the sleep loss is determined by the patient's sleep loss score. In some embodiments, the patient's sleep loss score after the anti-IL-13 antibody treatment is reduced for two points or greater compared to the patient's sleep score at baseline. In some embodiments, the induction period (or the first period) is 16 weeks, and during the induction period (or the first period), the anti-IL-13 antibody is administered at 500 mg at baseline (week 0) and week 2, followed by 250 mg once every two weeks for 14 weeks. In some embodiments, the maintenance period (or the second period) is 36 weeks. In some embodiments, the anti-IL-13 antibody is administered to the patient subcutaneously.

In some embodiments, the patient has moderate to severe atopic dermatitis for at least a year at the baseline. In some embodiments, the patient has an EASI score of 16 or greater, an IGA score of 3 or greater, and more than 10% of BSA affected by atopic dermatitis, at the baseline. In some embodiments, the patient has inadequate response to topical corticosteroids, topical calcineurin inhibitors, or crisaborole; or topical corticosteroids, topical calcineurin inhibitors, or crisaborole are medically inadvisable for the patient. In some embodiments, the patient is aged 12 years and older. In some embodiments, the patient is aged 18 years and older.

In some embodiments, the moderate to severe atopic dermatitis can be determined by criteria known in the art, e.g., Hanifin and Rajka criteria (Acta Derm Venereol (Stockh) 1980; Suppl 92:44-7); Rajka and Langeland criteria (Rajka G and Langeland T, Acta Derm Venereol (Stockh) 1989; 144 (Suppl): 13-4); or the American Academy of Dermatology Consensus Criteria for Chronic Atopic Dermatitis (Eichenfield L F, et al. J Am Acad Dermatol. 2014; 70(2): 338-351). In some embodiments, the moderate to severe atopic dermatitis is determined by the American Academy of Dermatology Consensus Criteria for Chronic Atopic Dermatitis. Under the criteria, the essential features of atopic dermatitis include pruritus; eczema (acute, subacute, chronic); typical morphology and age specific patterns; chronic or relapsing history. The typical morphology and age specific patterns include facial, neck, and extensor involvement in infants and children; current or previous flexural lesions in any age group; sparing of the groin and axillary regions. Other important features that add support to the diagnosis include early age of onset; atopy; personal and/or family history; immunoglobulin E reactivity; xerosis. The associated features that could help to suggest the diagnosis of atopic dermatitis but are nonspecific to be used for defining or detecting atopic dermatitis for research and epidemiologic studies: atypical vascular responses (e.g. facial pallor, white dermographism, delayed blanch response); keratosis pilaris/pityriasis alba/hyperlinear palms/icthyosis; ocular/periorbital changes; perifollicular accentuation/lichenification/prurigo lesions. On occasion, skin biopsy specimens or other tests (such as serum immunoglobulin E, potassium hydroxide preparation, patch testing, and/or genetic testing) may be helpful to rule out other or associated skin conditions. The exclusionary conditions include scabies; seborrheic dermatitis; contact dermatitis (irritant or allergic); icthyoses; cutaneous T-cell lymphoma; psoriasis; photosensitivity dermatoses; immune deficiency diseases; erythroderma of other causes.

The anti-IL-13 antibodies suitable for use in the methods and uses provided herein have been described previously, e.g., WO2005062967. In some embodiments, the anti-IL-13 antibody binds IL-13 with high affinity and blocks signaling through the active IL-4Ralpha/IL-13Ralpha1 heterodimer. In some embodiments, the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6. In some embodiments, the anti-IL-13 antibody comprises a VH comprising SEQ ID NO: 7, and a VL comprising SEQ ID NO: 8. In some embodiments, the anti-IL-13 antibody comprises a heavy chain comprising SEQ ID NO: 9, and a light chain comprising SEQ ID NO: 10. In some embodiments, the anti-IL-13 antibody is lebrikizumab. The amino acid sequences of lebrikizumab are provided in Table 1. C-terminal clipping of IgG antibodies could occur where one or two C-terminal amino acids are removed from the heavy chain of the IgG antibodies. For example, if a C-terminal lysine (K) is present, it may be truncated or clipped off from the heavy chain. A penultimate glycine (G) may also be truncated or clipped off from the heavy chain as well. Modification of N-terminal amino acid of IgG could also occur. For example, the N-terminal glutamine (Q) or glutamic acid (E) can cyclize into pyro-glutamate (pE) spontaneously. SEQ ID NO: 9 reflects these potential modifications of lebrikizumab heavy chain.

TABLE 1
Lebrikizumab Sequences

| SEQ ID | | |
|---|---|---|
| NO: | Description | Sequence |
| 1 | Lebrikizumab | AYSVN |
| | HCDR1 | |
| 2 | Lebrikizumab | MIWGDGKIVYNSALKS |
| | HCDR2 | |
| 3 | Lebrikizumab | DGYYPYAMDN |
| | HCDR3 | |
| 4 | Lebrikizumab | RASKSVDSYGNSFMH |
| | LCDR1 | |
| 5 | lebrikizumab | LASNLES |
| | LCDR2 | |
| 6 | Lebrikizumab | QQNNEDPRT |
| | LCDR3 | |
| 7 | Lebrikizumab | VTLRESGPALVKPTQTLTLTCTVSGFSLSAYSVN |
| | heavy chain | WIRQPPGKALEWLAMIWGDGKIVYNSALKSRLTI |
| | variable region | SKDTSKNQVVLTMTNMDPVDTATYYCAGDGYY |
| | (VH) | PYAMDNWGQGSLVTVSS |
| 8 | Lebrikizumab | DIVMTQSPDSLSVSLGERATINCRASKSVDSYGN |
| | light chain | SFMHWYQQKPGQPPKLLIYLASNLESGVPDRFSG |
| | variable region | SGSGTDFTLTISSLQAEDVAVYYCQQNNEDPRTF |
| | (VL) | GGGTKVEIK |
| 9 | Lebrikizumab | Xaa1VTLRESGPALVKPTQTLTLTCTVSGFSLSAYS |
| | heavy chain | VNWIRQPPGKALEWLAMIWGDGKIVYNSALKSR |
| | (HC) | LTISKDTSKNQVVLTMTNMDPVDTATYYCAGDG |
| | | YYPYAMDNWGQGSLVTVSSASTKGPSVFPLAPC |
| | | SRSTSESTAALGCLVKDYFPEPVTVSWNSGALTS |
| | | GVHTFPAVLQSSGLYSLSSVVTVPSSSLGTKTYTC |
| | | NVDHKPSNTKVDKRVESKYGPPCPPCPAPEFLGG |
| | | PSVFLFPPKPKDTLMISRTPEVTCVVVDVSQEDPE |
| | | VQFNWYVDGVEVHNAKTKPREEQFNSTYRVVS |
| | | VLTVLHQDWLNGKEYKCKVSNKGLPSSIEKTISK |
| | | AKGQPREPQVYTLPPSQEEMTKNQVSLTCLVKG |
| | | FYPSDIAVEWESNGQPENNYKTTPPVLDSDGSFF |
| | | LYSRLTVDKSRWQEGNVFSCSVMHEALHNHYTQ |
| | | KSLSLSLXaa2Xaa3 |
| | | wherein Xaa1 is Q, pE, or absent; Xaa2 is G or |
| | | absent; Xaa3 is K or absent. |
| 10 | Lebrikizumab | DIVMTQSPDSLSVSLGERATINCRASKSVDSYGN |
| | light chain | SFMHWYQQKPGQPPKLLIYLASNLESGVPDRFSG |
| | (LC) | SGSGTDFTLTISSLQAEDVAVYYCQQNNEDPRTF |
| | | GGGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLSSPVTKSFNRGEC |

The anti-IL-13 antibodies, e.g., lebrikizumab, can be formulated with suitable carriers or excipients into a pharmaceutical composition that is suitable for administration to patients. For example, the anti-IL-13 antibodies, e.g., lebrikizumab, can be formulated in a pharmaceutical composition as described in WO 2013/066866. The pharmaceutical composition can comprise 100 mg, 150 mg, 200 mg, 250 mg, 300 mg, 350 mg, 400 mg, 450 mg, or 500 mg of the anti-IL-13 antibody. In some embodiments, the pharmaceutical composition comprises 250 mg or 500 mg of the anti-IL-13 antibody. In some embodiments, the anti-IL-13 antibody concentration in the pharmaceutical composition is between 100 mg/mL and 150 mg/mL, e.g., 125 mg/mL. The pharmaceutical composition can also comprise 5 mM-40 mM histidine acetate buffer, pH 5.4 to 6.0. In some embodiments, the pharmaceutical composition further comprises a polyol (e.g., sugar) that has a concentration between 100 mM and 200 mM, and/or a surfactant (e.g., polysorbate 20) that has a concentration of 0.01%-0.1%. In one embodiment, the pharmaceutical composition comprises 125 mg/mL of anti-IL-13 antibody (e.g., lebrikizumab), 20 mM histidine acetate buffer, pH 5.7, 175 mM sucrose and 0.03% polysorbate 20.

In some embodiments, the anti-IL-13 antibody or a pharmaceutical composition comprising the anti-IL-13 antibody is administered subcutaneously to the patient. In some embodiments, the anti-IL-13 antibody or a pharmaceutical composition comprising the anti-IL-13 antibody is administered to the patient once every two weeks or once every four weeks. In some embodiments, the anti-IL-13 antibody or a pharmaceutical composition comprising the anti-IL-13 antibody is administered to the patient at 250 mg once every two weeks or once every four weeks. In some embodiments, the anti-IL-13 antibody or a pharmaceutical composition comprising the anti-IL-13 antibody is administered subcutaneously to the patient at 250 mg once every two weeks. In some embodiments, the anti-IL-13 antibody or a pharmaceutical composition comprising the anti-IL-13 antibody is administered subcutaneously to the patient at 250 mg once every four weeks.

In some embodiments, the anti-IL-13 antibody or a pharmaceutical composition comprising the anti-IL-13 antibody is administered to the patient using a subcutaneous administration device. The subcutaneous administration device can be selected from a prefilled syringe, disposable pen injection device, microneedle device, microinfuser device, needle-free injection device, or autoinjector device. Various subcutaneous administration devices, including autoinjector devices, are known in the art and are commercially available. Exemplary devices include, but are not limited to, prefilled syringes (such as BD HYPAK SCF®, READYFILL™ and STERIFILL SCF™ from Becton Dickinson; CLEARSHOT™ copolymer prefilled syringes from Baxter; and Daikyo Seiko CRYSTAL ZENITH® prefilled syringes available from West Pharmaceutical Services); disposable pen injection devices such as BD Pen from Becton Dickinson; ultra-sharp and microneedle devices (such as INJECT-EASE™ and microinfuser devices from Becton Dickinson; and H-PATCH™ available from Valeritas) as well as needle-free injection devices (such as BIOJECTOR® and IJECT® available from Bioject; and SOF-SERTER® and patch devices available from Medtronic). In some embodiments, the subcutaneous administration device is an autoinjector device described in WO 2008/112472, WO 2011/109205, WO 2014/062488, and/or WO 2016/089864.

In some embodiments, the patient can be treated with the anti-IL-13 antibody or a pharmaceutical composition comprising the anti-IL-13 antibody for a period of up to 52 weeks, e.g., about 4 to 52 weeks, about 4 weeks, about 6 weeks, about 8 weeks, about 10 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks.

In some embodiments, the patient is treated with the anti-IL-13 antibody or a pharmaceutical composition comprising the anti-IL-13 antibody for an induction period (or the first period) of up to 16 weeks (e.g., about 4 to 16 weeks, about 6 to 16 weeks, about 8 to 16 weeks, about 10 to 16 weeks, about 12 to 16 weeks, about 4 to 12 weeks, about 6 to 12 weeks, about 8 to 12 weeks, about 4 to 8 weeks, about 4 to 10 weeks, about 4 weeks, 6 weeks, 8 weeks, 10 weeks, 12 weeks, 14 weeks, 16 weeks). During the induction period (or the first period), the anti-IL-13 antibody is administered at a loading dose of 500 mg at baseline (week 0) and week 2, followed by a subsequent dose of 250 mg once every two weeks for 2 to 14 weeks (e.g., about 4 to 14 weeks, about 6 to 14 weeks, about 8 to 14 weeks, about 10 to 14 weeks, about 12 to 14 weeks, about 4 to 12 weeks, about 6 to 12 weeks, about 8 to 12 weeks, about 10 to 12 weeks, about 4 to 6 weeks, about 4 to 8 weeks, about 4 to 10 weeks, about 6 to 10 weeks, about 8 to 10 weeks, about 2 weeks, 4 weeks, 6 weeks, 8 weeks, 10 weeks, 12 weeks, 14 weeks). In some embodiments, the induction period (or the first period) is 4 to 16 weeks. In some embodiments, the induction period (or the first period) is 16 weeks. In such embodiments, during the induction period (or the first period), the anti-IL-13 antibody is administered at a loading dose of 500 mg at baseline (week 0) and week 2, followed by a subsequent dose of 250 mg once every two weeks for 14 weeks.

Before, during and after the treatment of the anti-IL-13 antibody, the patient can be assessed for one or more characteristics of the Atopic Dermatitis Disease Severity Measures (ADDSM), which determine certain signs, symptoms, features, or parameters that have been associated with atopic dermatitis and that can be quantitatively or qualitatively assessed. Exemplary ADDSM include, but are not limited to, Eczema Area and Severity Index (EASI), Investigator Global Assessment (IGA), body surface area (BSA), Scoring of Atopic Dermatitis (SCORAD), Pruritus Numerical Rating Scale (NRS), Sleep loss scale, Skin pain NRS score, Patient-Oriented Eczema Measure (POEM) total score, Dermatology Life Quality Index (DLQI) or Children Dermatology Life Quality Index (CDLQI), DLQI-Relevant (DLQI-R) score, Patient-Reported Outcomes Measurement Information System (PROMIS) Anxiety and Depressive Symptoms, EQ-5D (European Quality of Life-5 Dimensions), ACQ-5 (Asthma Control Questionnaire-5), World Health Organization-Five Well-Being Index (WHO-5) score, Recap of Atopic Eczema (RECAP) score, Treatment Satisfaction Questionnaire for Medication-9 items (TSQM-9) score. The ADDSM can be measured at baseline and at one or more time points after administration of the anti-IL13 antibody or pharmaceutical composition comprising the anti-IL-13 antibody. The difference between the value of the ADDSM at a particular time point following initiation of treatment and the value of the ADDSM at baseline is used to establish whether there has been an improvement (e.g., a reduction) in the ADDSM.

In some embodiments, the methods and therapeutic uses described herein further comprise determining one or more of the following characteristics of the patient at baseline and during and after the induction period (or the first period): the EASI score; the IGA score; the percentage of BSA affected by atopic dermatitis; pruritus NRS score; SCORAD score; sleep loss score; POEM total score; DLQI or CDLQI score; EQ-5D; ACQ-5; PROMIS Anxiety and Depressive Symptoms.

In some embodiments, the patient's EASI score is determined after the induction period (or the first period). In some embodiments, the patient's EASI score determined after the induction period (or the first period) is reduced by 50% or greater compared to the patient's EASI score at the baseline, which means the patient has achieved “EASI-50”. In some embodiments, the patient's EASI score determined after the induction period (or the first period) is reduced by 75% or greater compared to the patient's EASI score at the baseline, which means the patient has achieved “EASI-75”. In some embodiments, the patient's EASI score determined after the induction period (or the first period) is reduced by 90% or greater compared to the patient's EASI score at the baseline, which means the patient has achieved “EASI-90”. A patient is considered a responder to the anti-IL13 antibody when the patient reached EASI-75 after the induction period (or the first period).

In some embodiments, the patient's IGA score is determined after the induction period (or the first period). A patient is considered a responder to the anti-IL13 antibody when the patient's IGA score is 0 or 1 after the induction period (or the first period). In some embodiments, a patient is considered a responder to the anti-IL13 antibody when the patient's IGA score is 0 or 1 after the induction period (or the first period), and the patient's IGA score after the induction period (or the first period) is reduced by 2 points or greater compared to the patient's IGA score determined at the baseline.

After completion of the induction period (or the first period), the patient enters a maintenance period (or a second period). During the maintenance period (or the second period), the patient is further treated with the anti-IL13 antibody or a pharmaceutical composition comprising the anti-IL-13 antibody. The dosing regimen for the maintenance period (or the second period) can be selected based on the patient's ADDSM assessment and response to the IL-13 antibody after the induction period (or the first period), e.g., the patient's IGA or EASI score after the induction period (or the first period), and/or the patient's own characteristics, e.g., weight, age, race.

The maintenance period (or the second period) can be up to 36 weeks (e.g., about 4 to 36 weeks, about 8 to 36 weeks, about 12 to 36 weeks, about 16 to 36 weeks, about 20 to 36 weeks, about 24 to 36 weeks, about 28 to 36 weeks, about 4 to 32 weeks, about 8 to 32 weeks, about 12 to 32 weeks, about 16 to 32 weeks, about 20 to 32 weeks, about 24 to 32 weeks, about 28 to 32 weeks, about 4 to 24 weeks, about 8 to 24 weeks, about 12 to 24 weeks, about 16 to 24 weeks, about 20 to 24 weeks, about 4 to 20 weeks, about 8 to 20 weeks, about 12 to 20 weeks, about 16 to 20 weeks, about 4 to 16 weeks, about 8 to 16 weeks, about 12 to 16 weeks, about 4 to 12 weeks, about 8 to 12 weeks, about 4 weeks, about 8 weeks, about 12 weeks, about 16 weeks, about 20 weeks, about 24 weeks, about 28 weeks, about 32 weeks, about 36 weeks). In some embodiments, the maintenance period (or the second period) is 8 to 36 weeks. In some embodiments, the maintenance period (or the second period) is 36 weeks.

In some embodiments, the methods and therapeutic uses described herein further comprise determining one or more of the following characteristics of the patient during and after the maintenance period (or the second period): the EASI score; the IGA score; the percentage of BSA affected by atopic dermatitis; pruritus NRS score; SCORAD score; sleep loss score; POEM total score; DLQI or CDLQI score; EQ-5D; ACQ-5; PROMIS Anxiety and Depressive Symptoms. Similarly, the patient's EASI score during and after the maintenance period (or the second period) can be evaluated to see if the patient has reached EASI-50, EASI-75, or EASI-90. The patient's IGA score during and after the maintenance period (or the second period) can be evaluated to see if the patient's IGA score is 0 or 1 and if the patient's IGA score is reduced by 2 points or greater.

The “Investigator Global Assessment” or “IGA” is an assessment measure used globally to rate the severity of the patient's AD (Simpson E, et al. J Am Acad Dermatol. 2020; 83(3): 839-846). It is based on a 5-point scale ranging from 0 (clear) to 4 (severe) and a score is selected using descriptors that best describe the overall appearance of the lesions at a given time point (see Table 2). It is not necessary that all characteristics under Morphological Description be present. The IGA can be conducted prior to conducting the EASI and BSA assessments.

TABLE 2
Investigator Global Assessment (IGA)

| Score | Grade | Definition |
|---|---|---|
| 0 | Clear | Minor, residual discoloration; no erythema or |
| | | induration/papulation; no oozing/crusting; no edema. |
| 1 | Almost clear | Trace, faint pink erythema with barely perceptible |
| | | induration/papulation and no oozing/crusting; no edema. |
| 2 | Mild | Faint-pink erythema with papulation and edema perceptible |
| | | upon palpation and no oozing/crusting; minimal induration. |
| 3 | Moderate | Pink-red erythema with definite edema of skin papules and |
| | | plaques; there may be some oozing/crusting; palpable |
| | | induration. |
| 4 | Severe | Deep/bright red erythema with significant swelling and |
| | | obvious raised borders of papules and plaques with |
| | | oozing/crusting; significant induration. |

The “Eczema Area and Severity Index” or “EASI” is a measure used in clinical settings to assess the severity and extent of AD (Hanifin et al., Exp Dermatol. 2001; 10:11-18). EASI is a composite index with scores ranging from 0 to 72, with the higher values indicating more severe and or extensive disease. The severity of erythema, induration/papulation, excoriation, and lichenification can be assessed by a clinician or other medical professional on a scale of 0 (absent) to 3 (severe) for each of the 4 body areas: head and neck, trunk, upper limbs, and lower limbs, with half points allowed. In addition, the extent of AD involvement in each of the 4 body areas can be assessed as a percentage by body surface area of head, trunk, upper limbs, and lower limbs, and converted to a score of 0 to 6. A total score (0-72) is assigned based on the sum of total scores for each of the four body region scores.

The body surface area (BSA) assessment estimates the extent of disease or skin involvement with respect to AD and is expressed as a percentage of total body surface. BSA is determined by a clinician or other medical professional using the patient palm is about 1% BSA rule.

The “Scoring of Atopic Dermatitis” or “SCORAD” is a validated clinical tool for assessing the extent and intensity of AD developed by the European Task Force on Atopic Dermatitis (Consensus report of the European Task Force on Atopic Dermatitis. Dermatology. 1993; 186(1): 23-31). There are 3 components to the assessment: (i) the extent of AD is assessed as a percentage of each defined body area and reported as the sum of all areas, with a score ranging from 0 to 100 (assigned as “A” in the overall SCORAD calculation); (ii) the severity of 6 symptoms of AD: redness, swelling, oozing/crusting, excoriation, skin thickening/lichenification, dryness. Each item is graded as follows: none (0), mild (1), moderate (2), or severe (3) (for a maximum of 18 total points, assigned as “B” in the overall SCORAD calculation); (iii) subjective assessment of itch and of sleeplessness is recorded for each symptom using a visual analogue scale (VAS), where 0 is no itch (or sleeplessness) and 10 is the worst imaginable itch (or sleeplessness), with a maximum possible score of 20 (assigned as “C” in the overall SCORAD calculation). The SCORAD Index formula is: A/5+7B/2+C. The maximal score of the SCORAD Index is 103.

Pruritus Numerical Rating Scale (NRS) is an 11-point scale used by patients (and if applicable, with help of parents/caregiver if required) to rate their worst itch severity over the past 24 hours with 0 indicating “No itch” and 10 indicating “Worst itch imaginable” (Phan N Q, et al. Acta Derm Venereol 2012; 92:502-507). Assessments are recorded by the patient daily using an electronic diary. The baseline pruritus NRS is determined based on the average of daily Pruritus NRS during the 7 days immediately preceding baseline. A minimum of 4 daily scores out of the 7 days immediately preceding baseline is required for this calculation.

Sleep loss scale rates patient's sleep loss due to pruritus on a 5-point Likert scale (with scores ranging from 0 [not at all], 1 [a little], 2 [moderately], 3 [quite a bit], to 4 [unable to sleep at all]). Assessments will be recorded daily by the patient using an electronic diary.

The Skin Pain NRS is an 11-point scale completed by patients (and if applicable, with help of parents/caregiver if required) to rate their worst skin pain (for example discomfort or soreness) severity over the past 24 hours with 0 indicating “No pain” and 10 indicating “Worst pain imaginable” (Newton L, et al. J Patient Rep Outcomes. 2019 Jul. 16; 3:42). Assessments are recorded by the patient daily up until Week 16 and weekly from Week 16 onwards using an electronic diary. The baseline Skin Pain NRS is determined based on the average of daily Skin Pain NRS during the 7 days immediately preceding baseline. A minimum of 4 daily scores out of the 7 days immediately preceding baseline is required for this calculation.

The Patient-Oriented Eczema Measure (POEM) is a 7-item, validated, questionnaire completed by the patient (and if applicable, with help of parents/caregiver if required) to assess disease symptoms over the last week (Centre of Evidence Based Dermatology. POEM-Patient Oriented Eczema Measure. Available at https://www.nottingham.ac.uk/research/groups/cebd/resources/poem.aspx). Patients are asked to respond to 7 questions on skin dryness, itching, flaking, cracking, sleep loss, bleeding, and weeping. All 7 answers carry equal weight with a total possible score from 0 to 28 (answers scored as: No days=0; 1-2 days=1; 3-4 days=2; 5-6 days=3; everyday=4). A high score is indicative of a poor quality of life. POEM responses are captured weekly using an electronic diary.

The Dermatology Life Quality Index (DLQI) is a 10-item, validated questionnaire completed by the patient or caregiver, used to assess the impact of skin disease on the quality of life of the patient (Finlay, A. Y. and Khan, G. K. 1994. Clinical and Experimental Dermatology 1993 Sep. 23; 19:210-216). The 10 questions cover the following topics: symptoms, embarrassment, shopping and home care, clothes, social and leisure, sport, work or study, close relationships, sex, and treatment, over the previous week. Each question is scored from 0 to 3 (“not at all,” “a little,” “a lot,” and “very much”), giving a total score ranging from 0 to 30. A high score is indicative of a poor quality of life.

For adolescents below the age of 16, the Children DLQI (CDLQI) is employed which is based on a set of 10 questions different from those of the DLQI (Lewis-Jones M S, Finlay A Y. British Journal of Dermatology, 1995; 132:942-949).

The DLQI-Relevant (DLQI-R) is a recently developed scoring that adjusts the total score of the DLQI questionnaire for the number of not relevant responses (NRRs) indicated by a patient (Rencz F, et al. Br J Dermatol. 2020; 182(5): 1167-1175).

Patient-Reported Outcomes Measurement Information System (PROMIS) is a set of person-centered measures that evaluates and monitors physical, mental, and social health in adults and children. The PROMIS® measures used in this study include Anxiety and Depression short forms, which assess the patients' symptoms over the previous week. Patients≤17 years will complete pediatric versions for the duration of the study.

The PROMIS Anxiety Short Form v1.0—Anxiety 8a is a participant administered questionnaire that assesses the following items in adults: self-reported fear (fearfulness, panic); anxious misery (worry, dread); hyperarousal (tension, nervousness, restlessness), and somatic symptoms related to arousal (racing heart, dizziness) (PROMIS Anxiety 2019, Published Mar. 1, 2019. Accessed Mar. 8, 2021. Available at https://www.healthmeasures.net/images/PROMIS/manuals/PROMIS_Anxiety_Scoring_Manual.pdf). Each question has 5 response options, with scores ranging from 1 to 5. The total scores range from 8 to 40, with the higher score indicating a higher level of anxiety. The adult self-report assesses anxiety “in the past 7 days.”

The PROMIS Depression Short Form v1.0—Depression 8a is a participant administered questionnaire that assesses the following items in adults: self-reported negative mood (sadness, guilt); views of self (self-criticism, worthlessness); social cognition (loneliness, interpersonal alienation), and decreased positive affect and engagement (loss of interest, meaning, and purpose) (PROMIS Depression 2019, Published Feb. 28, 2019. Accessed Mar. 8, 2021. Available at https://www.healthmeasures.net/images/PROMIS/manuals/PROMIS_Depression_Scoring_Manual.pdf). Somatic symptoms (such as changes in appetite or sleeping patterns) are not included. This helps eliminate potential confounding effects of these items when assessing participants with co-morbid physical conditions. Each question has 5 response options, with scores ranging from 1 to 5. The total scores range from 8 to 40, with the higher score indicating a higher level of depression. The adult self-report assesses depression “in the past 7 days.”

EQ-5D (European Quality of Life-5 Dimensions) comprises five dimensions: mobility, self-care, usual activities, pain/discomfort and anxiety/depression. The EQ VAS records the patient's self-rated health on a vertical visual analogue scale. The scores on these five dimensions can be presented as a health profile or can be converted to a single summary index number (utility) reflecting preferability compared to other health profiles. EQ-5D is completed by the patient in the study clinic.

The European Quality of Life-5 Dimensions-5 Levels (EuroQol-5D-5L or EQ-5D-5L) is a participant-administered, 5 questions plus 1 visual analog scale (VAS) standardized measure of health status in adults that provides a simple, generic measure of health for clinical and economic appraisal. The EQ-5D-5L consists of 2 components: a descriptive system of the respondent's health and a rating of his or her current health state using a 0 to 100 mm VAS (20 cm). The descriptive system comprises the following 5 dimensions: mobility, self care, usual activities, pain/discomfort, and anxiety/depression. Each dimension has 5 levels: no problems, slight problems, moderate problems, severe problems, and extreme problems. The respondent is asked to indicate his or her health state by ticking (or placing a cross) in the box associated with the most appropriate statement in each of the 5 dimensions. It should be noted that the numerals 1 to 5 have no arithmetic properties and should not be used as an ordinal score. The EQ-5D-5L health states, defined by the EQ-5D-5L descriptive system, may be converted into a single summary index by applying a formula that essentially attaches values (also called weights) to each of the levels in each dimension. The VAS records the respondent's self-rated health on a vertical VAS where the endpoints are labeled “best imaginable health state” and “worst imaginable health state.” This information can be used as a quantitative measure of health outcome (Herdman et al., Qual Life Res. 2011; 20(10): 1727-1736; EuroQol Group, EQ-5D-5L User Guide. Version 2.1. April 2015. Accessed: Jan. 14, 2021. Available at https://euroqol.org/wp-content/uploads/2016/09/EQ-5D-5L_UserGuide_2015.pdf). The self-rated health status captured by EQ-5D-5L relates to the participant's situation at the time of completion. No attempt is made to recall health status over the preceding days or weeks (EuroQol Group 2015).

ACQ-5 is Asthma Control Questionnaire. Patients who report comorbid asthma prior to enrollment will complete the Asthma Control Questionnaire (ACQ-5) in addition to other patient reported outcomes in this trial. The ACQ-5 has been shown to reliably measure asthma control and distinguish patients with well-controlled asthma (score ≤0.75 points) from those with uncontrolled asthma (score ≥1.5 points). It consists of 5 questions that are scored on a 7-point Likert scale with a recall period of 1 week. The total ACQ-5 score is the mean score of all questions; a lower score represents better asthma control. ACQ-5 is completed by the patient in the study clinic.

In another aspect, provided herein are an anti-IL-13 antibody or a pharmaceutical composition comprising an anti-IL-13 antibody for use in the treatment of moderate to severe atopic dermatitis in a patient.

In another aspect, provided herein are uses of an anti-IL-13 antibody in the manufacture of a medicament for the treatment of moderate to severe atopic dermatitis in a patient.

In some embodiments, the methods and uses described herein further comprise administrating one or more topical corticosteroids to the patient. Exemplary topical corticosteroids include, but are not limited to, triamcinolone acetonide, hydrocortisone, and a combination of triamcinolone acetonide and hydrocortisone. Triamcinolone acetonide is typically formulated at a concentration of 0.1% in a cream, and hydrocortisone is typically formulated at a concentration of 1% or 2.5% in a cream. Certain topical corticosteroids are considered very high potency such as, for example, betamethasone dipropionate, clobetasol propionate, diflorasone diacetate, fluocinonide, and halobetasol propionate. Certain topical corticosteroids are considered high potency such as, for example, amcinonide, desoximetasone, halcinonide, and triamcinolone acetonide. Certain topical corticosteroids are considered medium potency, such as, for example, betamethasone valerate, clocortolone pivalate, fluocinolone acetonide, flurandrenolide, fluocinonide, fluticasone propionate, hydrocortisone butyrate, hydrocortisone valerate, mometasone furoate, and prednicarbate. Certain topical corticosteroids are considered low potency, such as, for example, alclometasone dipropionate, desonide, and hydrocortisone. TCS may be applied to affected areas once daily, twice daily, three times per day, or as needed. In some embodiments, the patient is inadequately controlled on topical corticosteroids. In some embodiments, the topical corticosteroid is triamcinolone acetonide, hydrocortisone, or a combination of triamcinolone acetonide and hydrocortisone. In some embodiments, the topical corticosteroids are administered concomitantly or sequentially with the anti-IL-13 antibody. In some embodiments, the topical corticosteroids are administered concomitantly with the anti-IL-13 antibody.

As used herein, the term “a,” “an,” “the” and similar terms used in the context of the present disclosure (especially in the context of the claims) are to be construed to cover both the singular and plural unless otherwise indicated herein or clearly contradicted by the context.

The term “about” as used herein, means in reasonable vicinity of the stated numerical value, such as plus or minus 10% of the stated numerical value.

The term “antibody,” as used herein, refers to an immunoglobulin molecule that binds an antigen. Embodiments of an antibody include a monoclonal antibody, polyclonal antibody, human antibody, humanized antibody, chimeric antibody, or conjugated antibody. The antibodies can be of any class (e.g., IgG, IgE, IgM, IgD, IgA) and any subclass (e.g., IgG1, IgG2, IgG3, IgG4).

An exemplary antibody is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Exemplary embodiments of antibodies of the present disclosure also include antibody fragments or antigen-binding fragments, which comprise at least a portion of an antibody retaining the ability to specifically interact with an antigen such as Fab, Fab′, F(ab′) 2, Fv fragments, scFv, scFab, disulfide-linked Fvs (sdFv), a Fd fragment and linear antibodies.

The term “baseline”, as used herein, means prior to or at the time of administration of the first dose (week 0) of the anti-IL-13 antibody or a pharmaceutical composition comprising the anti-IL-13 antibody. For example, the numerical value of an Atopic Dermatitis Disease Severity Measure (ADDSM) prior to or at the time of administration of the first dose of the anti-IL-13 antibody or a pharmaceutical composition comprising the anti-IL-13 antibody is considered the baseline value for that ADDSM.

The terms “bind” and “binds”, as used herein are intended to mean, unless indicated otherwise, the ability of a protein or molecule to form a chemical bond or attractive interaction with another protein or molecule, which results in proximity of the two proteins or molecules as determined by common methods known in the art.

The term “flare” as used herein refers to increase in signs and/or symptoms leading to escalation of therapy, which can be an increase in dose, a switch to a higher-potency class of drug, or the start of another drug.

The term “high affinity” as used herein refers to the strength of binding of an antibody to human IL-13 with an equilibrium dissociation constant (K_{D}) of less than about 10⁻⁸ M, e.g., from 10⁻¹⁵ M to 10⁻⁸ M, or from 10⁻¹² M to 10⁻⁹ M.

The term “human IL-13” refers to human interleukin 13 (also known as P600), an immunoregulatory cytokine produced primarily by activated Th2 cells. There are two known human IL-13 isoforms: isoform a and isoform b. The term “human IL-13” as used herein refers collectively to all human IL-13 isoforms. The amino acid sequence for human IL-13 isoform a can be found at NCBI Accession No. NP_002179.2. The amino acid sequence for human IL-13 isoform b can be found at NCBI Accession No. NP 001341922.1.

The term “inadequate response” as used herein refers to inability to achieve good disease control of atopic dermatitis (e.g., not able to achieve IGA≤2 or EASI 75) after use of the treatment for the duration recommended by the product prescribing information, or flare of atopic dermatitis occurs while on the treatment.

The term “intolerance” or “intolerant” as used herein refers to unacceptable toxicity (e.g., elevated creatinine, elevated liver function tests, uncontrolled hypertension, paranesthesia, headache, nausea, hypertrichosis), or requirement for a drug at doses or duration beyond those specified in the prescribing information.

The term “patient”, as used herein, refers to a human patient.

The term “topical corticosteroid” or “TCS”, as used herein includes Group I, Group II, Group III and Group IV topical corticosteroids. According to the Anatomical Therapeutic Chemical (ATC) Classification System of World Health Organization, the corticosteroids are classified as weak (Group I), moderately potent (Group II) and potent (Group III) and very potent (Group IV), based on their activity as compared to hydrocortisone. Group IV TCS (very potent) are up to 600 times as potent as hydrocortisone and include clobetasol and halcinonide. Group III TCS (potent) are 50 to 100 times as potent as hydrocortisone and include, but are not limited to, betamethasone valerate, betamethasone dipropionate, diflucortolone valerate, hydrocortisone-17-butyrate, mometasone furoate, and methylprednisolone aceponate. Group II TCS (moderately potent) are 2 to 25 times as potent as hydrocortisone and include, but are not limited to, clobetasone butyrate, and triamcinolone acetonide. Group I TCS (weak or mild) includes hydrocortisone, prednisolone, and methylprednisolone.

The term “topical calcineurin inhibitor” or “TCI”, as used herein, includes pimecrolimus, tacrolimus, and other inhibitors that suppress calcineurin activities and can be topically applied to a patient's skin.

As used herein, “treat”, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

### EXAMPLES

### Example 1. Two Randomized, Double-Blind, Placebo-Controlled Trials to Evaluate the Efficacy and Safety of Lebrikizumab in Patients with Moderate-to-Severe Atopic Dermatitis

Two identical Phase 3, randomized, double-blind, placebo-controlled, parallel-group studies were conducted to evaluate the safety and efficacy of lebrikizumab as monotherapy for moderate-to-severe atopic dermatitis (ADvocate 1 and ADvocate 2, i.e., NCT04146363 and NCT04178967). Each trial is 52 weeks in duration, including a 16-week induction period (or the first period) and a 36-week maintenance period (or the second period).

### Patient Population

Eligible adult and adolescent (≥12 to <18 years weighing ≥40 kg) patients with moderate-to severe atopic dermatitis for at least one year, defined according to the American Academy of Dermatology Consensus Criteria, an Eczema Area and Severity Index Score (EASI) of ≥16, an Investigator Global Assessment (IGA) score of ≥3 and a body surface area (BSA) of ≥10% are enrolled.

Inclusion Criteria: Patients must meet all the following criteria to be eligible for the study:
1. Adults and adolescents (≥12 to <18 years of age and weighing ≥40 kg).
2. Chronic AD (according to American Academy of Dermatology Consensus Criteria for Chronic Atopic Dermatitis) that has been present for ≥1 year before the screening visit.
3. Eczema Area and Severity Index (EASI) score ≥16 at the baseline visit.
4. Investigator Global Assessment (IGA) score ≥3 (scale of 0 to 4) at the baseline visit.
5. ≥10% body surface area (BSA) of AD involvement at the baseline visit.
6. History of inadequate response to treatment with topical medications; or determination that topical treatments are otherwise medically inadvisable.
7. Apply a stable dose of non-medicated topical moisturizer at least twice daily for ≥7 days prior to the baseline visit.
8. Completed electronic diary entries for pruritus and sleep-loss for a minimum of 4 of 7 days preceding randomization.
9. Willing and able to comply with all clinic visits and study-related procedures and questionnaires.
10. For women of childbearing potential: agree to remain abstinent or use a highly effective contraceptive method during the treatment period and for at least 18 weeks after the last dose of lebrikizumab or placebo.
11. Male patients must agree to use an effective barrier method of contraception during the study and for a minimum of 18 weeks following the last dose of study drug if sexually active with a female of childbearing potential.
12. Provide signed informed consent/assent.

Exclusion Criteria: Patients meeting any of the criteria below are excluded from the study:
1. Participation in a prior lebrikizumab clinical study.
2. History of anaphylaxis as defined by the Sampson criteria (Sampson et al., J Allergy Clin Immunol. 2006; 117(2): 391-397).
3. Treatment with topical corticosteroids, calcineurin inhibitors or phosphodiesterase-4 inhibitors such as crisaborole within 1 week prior to the baseline visit.
4. Prior treatment with dupilumab or tralokinumab.
5. Treatment with any of the following agents within 4 weeks prior to the baseline visit:
   a. Immunosuppressive/immunomodulating drugs (e.g., systemic corticosteroids, cyclosporine, mycophenolate-mofetil, IFN-γ, Janus kinase inhibitors, azathioprine, methotrexate);
   b. Phototherapy and photochemotherapy (PUVA) for AD.
6. Treatment with the following prior to the baseline visit:
   a. An investigational drug within 8 weeks or within 5 half-lives (if known), whichever is longer.
   b. B Cell-depleting biologics, including rituximab, within 6 months.
   c. Other biologics within 5 half-lives (if known) or 16 weeks, whichever is longer.
7. Use of prescription moisturizers within 7 days of the baseline visit.
8. Regular use (more than 2 visits per week) of a tanning booth/parlor within 4 weeks of the screening visit.
9. Treatment with a live (attenuated) vaccine within 12 weeks of the baseline visit or planned during the study.
10. Uncontrolled chronic disease that might require bursts of oral corticosteroids, e.g., co-morbid severe uncontrolled asthma (defined by an ACQ-5 score ≥1.5 or a history of ≥2 asthma exacerbations within the last 12 months requiring systemic [oral and/or parenteral] corticosteroid treatment or hospitalization for >24 hours).
11. Active chronic or acute infection requiring treatment with systemic antibiotics, antivirals, antiparasitics, antiprotozoals, or antifungals within 2 weeks before the baseline visit, or superficial skin infections within 1 week before the baseline visit.
12. Evidence of active acute or chronic hepatitis (as defined by the Department of Health & Human Services Centers for Disease Control and Prevention) or known liver cirrhosis.
13. Diagnosed active endoparasitic infections or at high risk of these infections.
14. Known or suspected history of immunosuppression, including history of invasive opportunistic infections (e.g., tuberculosis [TB], histoplasmosis, listeriosis, coccidioidomycosis, pneumocystosis, and aspergillosis) despite infection resolution: or unusually frequent, recurrent, or prolonged infections, per the Investigator's judgment.
15. History of human immunodeficiency virus (HIV) infection or positive HIV serology at screening.
16. In the Investigator's opinion, any clinically significant laboratory results from the chemistry, hematology or urinalysis tests obtained at the screening visit.
17. Presence of skin comorbidities that may interfere with study assessments.
18. History of malignancy, including mycosis fungoides, within 5 years before the screening visit, except completely treated in situ carcinoma of the cervix, completely treated and resolved non-metastatic squamous or basal cell carcinoma of the skin.
19. Severe concomitant illness(es) that in the Investigator's judgment would adversely affect the patient's participation in the study. Any other medical or psychological condition that in the opinion of the Investigator may suggest a new and/or insufficiently understood disease, may present an unreasonable risk to the study patient because of his/her participation in this clinical trial, may make patient's participation unreliable, or may interfere with study assessments.
20. Pregnant or breastfeeding women, or women planning to become pregnant or breastfeed during the study.

### Study Drug:

Pharmaceutical compositions containing 125 mg/mL lebrikizumab or placebo are supplied as sterile pre-filled syringes with a pre-assembled needle safety device (PFS-NSD) for subcutaneous administration to the patients. Lebrikizumab sequences are provided in Table 1. The placebo solution is identical in appearance and volume to the active solution except that it does not contain lebrikizumab.

### Study Design:

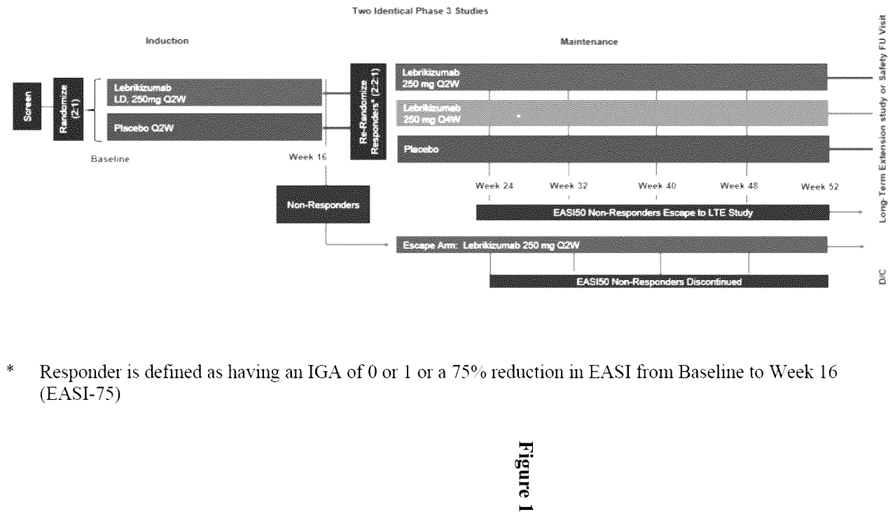

The study design in shown in FIG. 1.

In each trial, during the 16-week Induction Period (or the first period), approximately 400 patients are stratified and randomized 2:1 to either 250 mg lebrikizumab (loading dose of 500 mg given at Baseline (Week 0) and Week 2) or placebo by subcutaneous (SC) injection every 2 weeks (Q2W). All study drug injections are administered in the clinic.

After completion of the Week 16 visit, patients who have responded to treatment [defined as having an IGA of 0 or 1, or a 75% reduction in EASI from Baseline to Week 16 (EASI-75)] enter the Maintenance Period (or the second period) and are re-randomized 2:2:1 to one of the following treatment groups: lebrikizumab 250 mg once every two weeks (Q2W), lebrikizumab 250 mg once every four weeks (Q4W), or placebo Q2W. Patients are instructed to self-administer study drug at home.

Responders who received placebo during the first 16 weeks of the study and who are rerandomized to lebrikizumab arms receive a loading dose of lebrikizumab of either 500 mg given at Week 16 or 500 mg given at Weeks 16 and 18, based on the active treatment group assigned in Maintenance.

Patients who do not achieve an IGA of 0 or 1 or an EASI-75 at Week 16, and those patients not maintaining an EASI-50 response following re-randomization at Week 24, 32, 40 or 48, are assigned to an Escape Arm and receive lebrikizumab 250 mg Q2W as long-term treatment through Week 52. Patients not achieving an EASI-50 response after 8 weeks of treatment in the Escape Arm are terminated from the study.

Efficacy are measured through the IGA, EASI, BSA, SCORAD, Pruritus and Sleep-loss scores.

Safety are assessed by monitoring adverse events, serum chemistry, hematology and urinalysis laboratory testing, physical examination, pulse and blood pressure. An independent Data Safety Monitoring Board monitor patient safety by conducting formal reviews of accumulated safety data periodically throughout the trial. Additionally, adolescents are monitored for hormones.

Quality of life and impact of disease are assessed using the POEM, DLQI/CDLQI, EQ-5D and PROMIS® Anxiety and Depression measures. Patients reporting comorbid asthma at study entry complete the ACQ-5.

Serum samples are collected for pharmacokinetic analysis and immunogenicity.

Patients completing this 52-week study are offered the option of continued treatment in a separate long-term extension study. Patients who early terminate or choose not to enter the long-term extension study undergo a safety follow-up visit approximately 12 weeks after the last study drug injection.

### Objectives and Endpoints

The primary objective of this study is to evaluate the safety and efficacy of lebrikizumab compared with placebo in patients with moderate-to-severe AD.

For the U.S., the primary efficacy endpoint is the percentage of patients with an IGA score of 0 or 1 and a reduction ≥2 points from baseline to Week 16. The secondary objectives include: (1) Percentage of patients achieving EASI-75 (≥75% reduction from Baseline in EASI score) at Week 16; (2) Percentage of patients achieving EASI-90 (≥90% reduction from Baseline in EASI score) at Week 16; (3) Percentage change in Pruritus Numerical Rating Scale (NRS) score from Baseline to Week 16; (4) Percentage of patients with a Pruritus NRS of ≥4-points at Baseline who achieve a ≥4-point reduction from Baseline to Week 16; (5) Percentage change in EASI score from Baseline to Week 16; (6) Change from Baseline to Week 16 in percent BSA; (7) Percentage of patients achieving EASI-90 at Week 4; (8) Percentage change in Sleep-loss score from Baseline to Week 16; (9) Change from Baseline in Sleep-loss score at Week 16; (10) Percentage of patients with a Pruritus NRS of ≥4-points at Baseline who achieve a ≥4-point reduction from Baseline to Week 4: (11) Percentage of patients with a Pruritus NRS of ≥4-points at Baseline who achieve a ≥4-point reduction from Baseline to Week 2; (12) Percentage of patients with a Pruritus NRS of ≥4-points at Baseline who achieve a ≥4-point reduction from Baseline to Week 1. For the maintenance period, the secondary objectives include: (1) Percentage of patients from those rerandomized having achieved EASI-75 at Week 16 who continue to exhibit EASI-75 at Week 52 (EASI-75 calculated relative to baseline EASI score); (2) Percentage of patients from those rerandomized having achieved IGA 0 or 1 and a ≥2-point improvement from Baseline at Week 16 who continue to exhibit an IGA 0 or 1 and a ≥2-point improvement from Baseline at Week 52.

For Europe, the co-primary endpoints are: (1) the percentage of patients with an IGA score of 0 or 1 and a reduction ≥2 points from baseline to Week 16; and (2) the percentage of patients achieving EASI-75 (≥75% reduction from Baseline in EASI score) at Week 16. The secondary objectives include: (1) Percentage of patients achieving EASI-90 (≥90% reduction from Baseline in EASI score) at Week 16; (2) Percentage change in Pruritus Numerical Rating Scale (NRS) score from Baseline to Week 16; (3) Percentage of patients with a Pruritus NRS of ≥5-points at Baseline who achieve a ≥4-point reduction from Baseline to Week 16; (4) Percentage of patients with a Pruritus NRS of ≥4-points at Baseline who achieve a ≥4-point reduction from Baseline to Week 16; (5) Percentage change in EASI score from Baseline to Week 16; (6) Percentage of patients achieving EASI-90 at Week 4; (7) Change from baseline in DLQI at Week 16; (8) Percentage of patients achieving ≥4-point improvement in DLQI from baseline to Week 16; (9) Percentage change in Sleep-loss score from Baseline to Week 16; (10) Change from Baseline in Sleep-loss score at Week 16; (11) Percentage of patients with a Pruritus NRS of ≥5-points at Baseline who achieve a ≥4-point reduction from Baseline to Weeks 1, 2 and 4; (12) Percentage of patients with a Pruritus NRS of ≥4-points at Baseline who achieve a ≥4-point reduction from Baseline to Weeks 1, 2 and 4. For the maintenance period, the secondary objectives include: (1) Percentage of patients from those re-randomized having achieved EASI-75 at Week 16 who continue to exhibit EASI-75 at Week 52 (EASI-75 calculated relative to baseline EASI score); (2) Percentage of patients from those re-randomized having achieved IGA 0 or 1 and a ≥2-point improvement from Baseline at Week 16 who continue to exhibit an IGA 0 or 1 and a ≥2-point improvement from Baseline at Week 52; (3) Percentage of patients from those with a Pruritus NRS of ≥4-points at baseline re-randomized having achieved ≥4-point reduction from baseline at Week 16 who continue to exhibit ≥4-point reduction from baseline at Week 52; (4) Percentage of patients from those with a Pruritus NRS of ≥5-points at baseline re-randomized having achieved ≥4-point reduction from baseline at Week 16 who continue to exhibit ≥4-point reduction from baseline at Week 52; (5) Percentage change in SCORAD (having achieved EASI-75 at Week 16) from baseline at Week 52.

To evaluate the pharmacokinetics of lebrikizumab, average serum lebrikizumab concentration is measured.

Other secondary endpoints include: Proportion of patients with EASI-75, EASI-90 and EASI-50 by visit; Proportion of patients with IGA Score of 0 or 1 and a reduction ≥2 points from Baseline by visit; Percentage change from Baseline in EASI Score by visit; Percentage change from Baseline in Pruritus NRS by visit; Percentage of patients with Pruritus NRS change of ≥4 from Baseline by visit; Percentage of patients with a Pruritus NRS score of ≥4 points at Baseline who achieve a ≥4-point reduction from Baseline by visit; Change from Baseline in Sleep-Loss score by visit; Change from Baseline in DLQI/CDLQI by visit; Change from Baseline in EQ5D by visit; Change from Baseline in POEM by visit; Change from Baseline in PROMIS Anxiety measure by visit; Change from Baseline in PROMIS Depression measure by visit; Change in ACQ-5 score from Baseline to Week 16 in patients who have self-reported comorbid asthma; Percentage change from Baseline to Week 16 in SCORAD.

Statistical analyses are performed for the primary and secondary endpoints. The estimands and missing data imputation methods include Markov Chain Monte Carlo Multiple Imputation (MCMC-MI) and non-responder imputation (NRI).

### Results

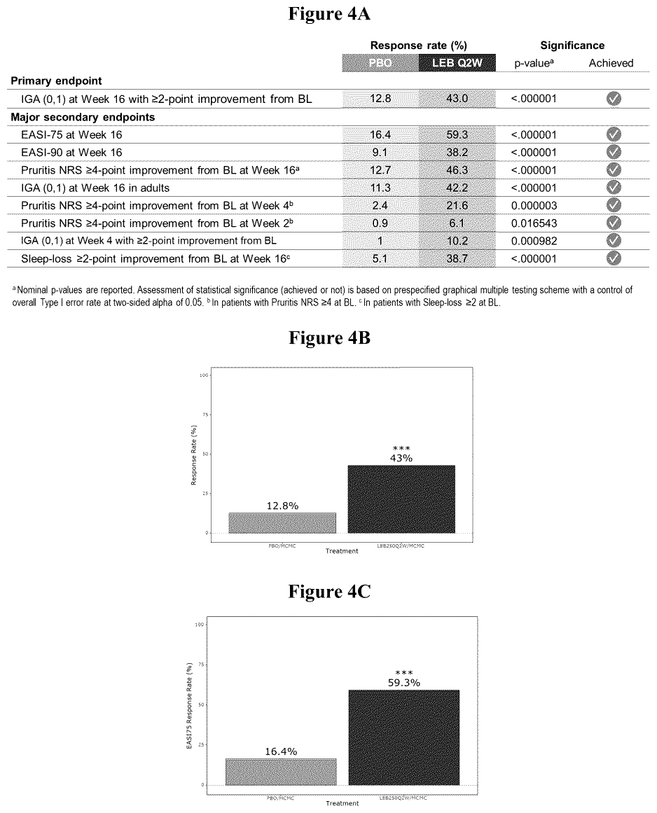

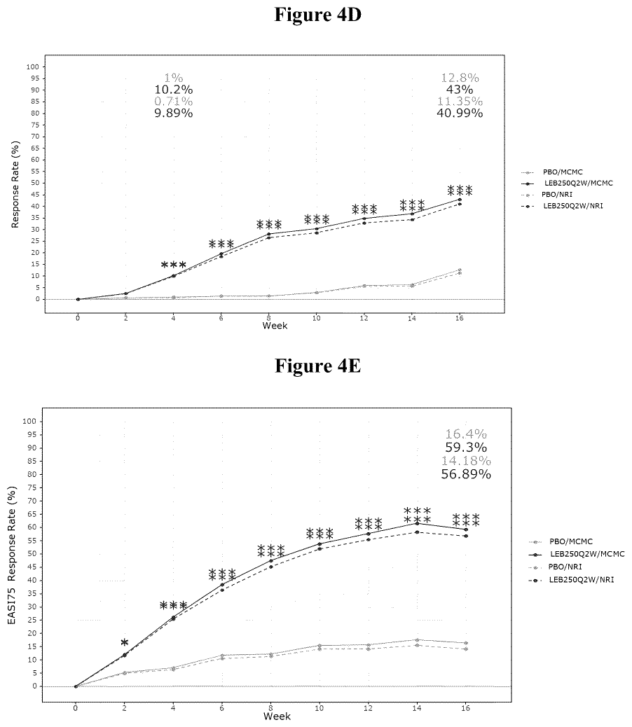

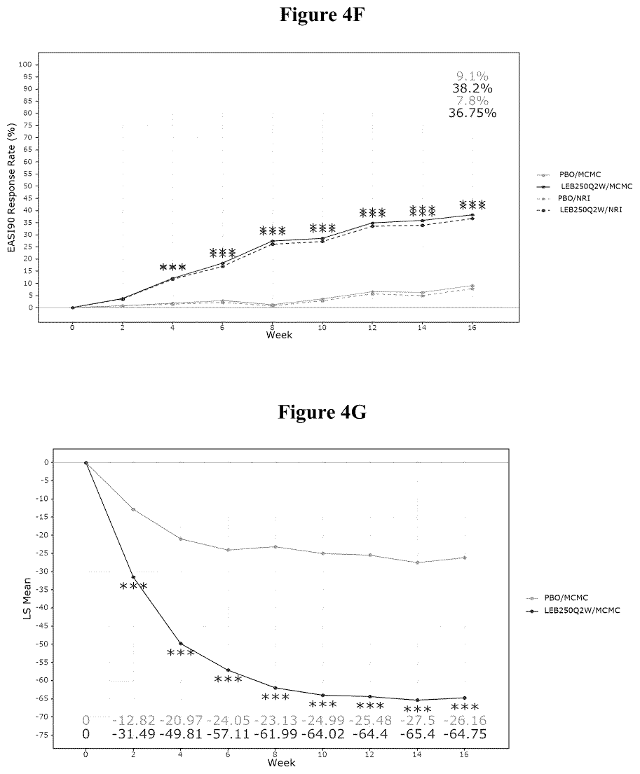

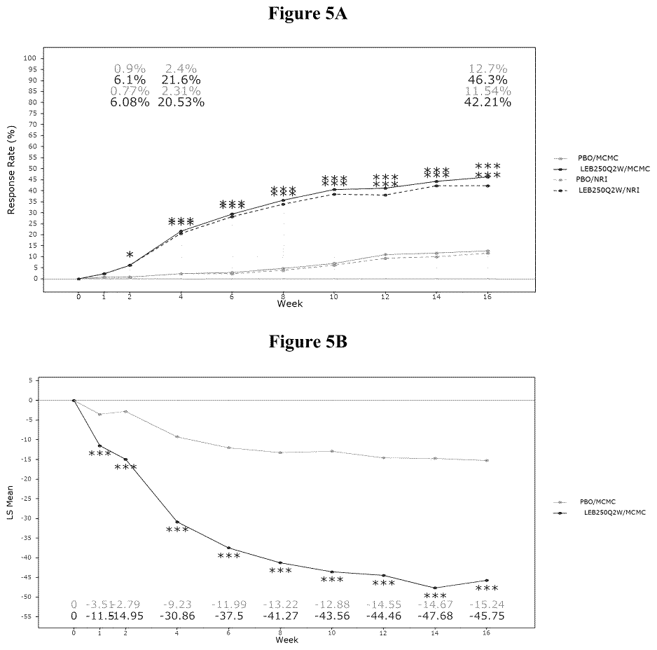

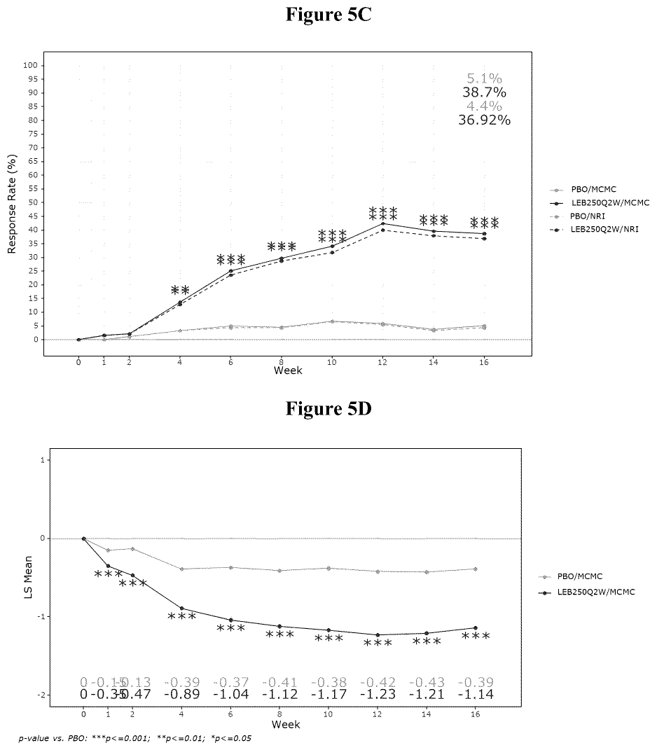

In the ADvocate 1 trial, all primary endpoints and key secondary endpoints, including skin and itch improvement, were met at Week 16 (FIGS. 4A-4G and 5A-5F).

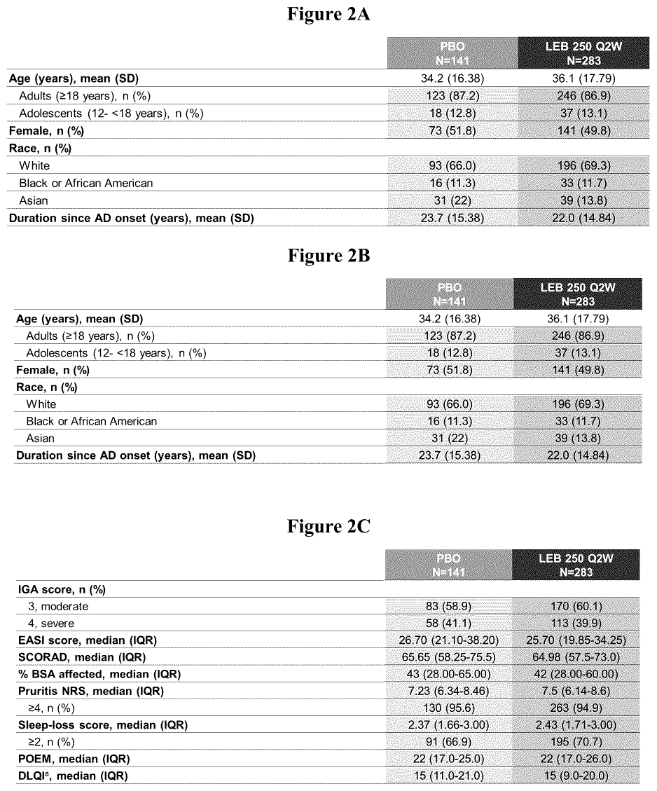

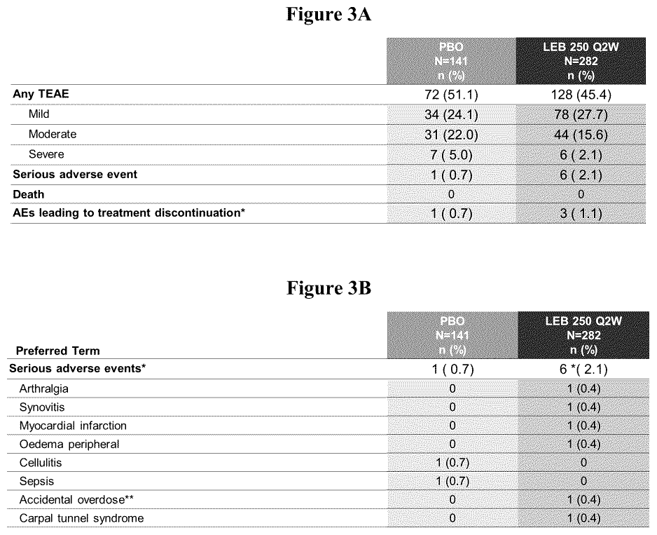

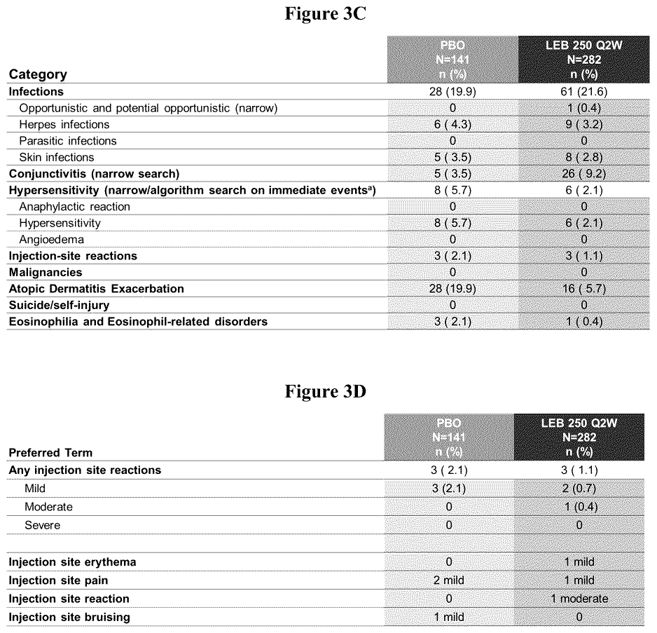

FIGS. 2A-2C show baseline demographics and baseline disease characteristics of the participants in ADvocate 1. FIGS. 3A-3C shows the adverse events through Week 16. FIG. 3D shows the injection site reactions through Week 16.

Compared to the placebo group, a statistically significant higher percentage of participants in the lebrikizumab treated group achieved skin clearance and skin improvement as early as Week 4 as measured by IGA 0/1, EASI-75, and EASI-90 (see FIGS. 4D-4G). A statistically significant higher percentage of participants in the lebrikizumab treated group achieved improvement in itch as early as Week 2, as measured by Pruritus NRS (FIGS. 5A-5B). A statistically significant higher percentage of participants in the lebrikizumab treated group experienced improvement in sleep and quality of life, as measured by the Sleep Loss score and DLQI scales, respectively (FIGS. 5C-5F).

In the Week 16 evaluation, lebrikizumab remained well tolerated with a comparable frequency of adverse events to the placebo group, including low frequency of injection site reactions. The overall frequencies of SAEs and discontinuation due to AE were low, with no deaths.

Similar results were seen in the ADvocate 2 trial, with all primary endpoints and key secondary endpoints, including skin and itch improvement, met at Week 16.

Therefore, lebrikizumab treatment achieved the primary and all key secondary endpoints, including itch, interference of itch on sleep and quality of life at Week 16, in two pivotal Phase 3 clinical trials.

Based on an interim analysis of Week 16 data, in ADvocate 1, the proportions of patients treated with lebrikizumab 250 mg (N=283) and placebo (N=141) achieving IGA 0/1 at Week 16 were 43.0% and 12.8% (p<0.001); EASI-75 responses were 59.3% and 16.4% (p<0.001); Pruritus NRS ≥4-point improvement from baseline (P≥4) proportions were 46.3% and 12.7% (p<0.001), respectively. The mean DLQI score at baseline in patients treated with lebrikizumab 250 mg (N=283) and placebo (N=141) was 15.3 and 15.7, respectively. Corresponding baseline means for EQ-5D VAS score were 68.2 and 67.0; for EQ-5D-5L US Health State Index were 0.7 and 0.7, respectively. At Week 16, the proportion of patients with ≥4-point improvement from baseline in DLQI score at Week 16 was 71.2% and 29.3% in lebrikizumab and placebo groups, among those patients with baseline DLQI score ≥4, respectively. The proportion of patients receiving lebrikizumab and placebo with DLQI (0,1) response was 26.3% and 4.2%, among those patients with baseline DLQI>1, respectively. The DLQI total score mean CFB at Week 16 was improved by −10.0 for lebrikizumab-treated patients and by −4.4 for placebo-treated patients. Statistical significance was achieved as early as Week 4, the first assessment after baseline, and continued through Week 16 for all DLQI analyses. There was also a significant difference between EQ-5D VAS score mean CFB (10.5 and 2.2, respectively) and EQ-5D-5L US Health State Index CFB (0.13 and 0.03, respectively) at Week 16 for patients assigned to lebrikizumab and placebo.

Based on an interim analysis of Week 16 data, in ADvocate2 (lebrikizumab, N=281, placebo, N=146), corresponding proportions for IGA 0/1 were 33.1% and 10.9% (p<0.001); EASI-75 responses were 50.8% and 18.2% (p<0.001); Pruritus NRS≥4-point improvement from baseline (P≥4) proportions were 38.3% and 11.3% (p<0.001), respectively. The baseline means for DLQI score were 15.4 and 15.9; EQ-5D VAS score were 66.7 and 68.6; EQ-5D-5L US Health State Index were 0.8 and 0.7, respectively. At Week 16, proportion of patients with ≥4-point improvement from baseline in DLQI score at Week 16 was 60.5% and 31.3% in lebrikizumab and placebo groups, among those patients with baseline DLQI score ≥4, respectively. The proportion of patients assigned to lebrikizumab and placebo achieving DLQI (0,1) response was 16.1% and 7.7%, among those patients with baseline DLQI>1, respectively. The DLQI total score mean CFB at Week 16 was improved by −9.3 for lebrikizumab-treated patients and by −4.9 for placebo-treated patients. Statistical significance was achieved as early as Week 4, the first assessment after baseline, and continued through Week 16 for DLQI ≥4-point improvement and total score CFB. Significant differences were also observed at Week 16 for EQ-5D VAS score mean CFB (9.0 and 5.2, respectively) and EQ-5D-5L US Health State Index CFB (0.08 and 0.03, respectively) for patients receiving lebrikizumab and placebo.

The percentage of patients reporting ≥1 TEAE was comparable in ADvocate1 (lebrikizumab, 45.4%; placebo, 51.1%) and ADvocate2 (lebrikizumab 53.0%; placebo, 66.2%).

After the final database lock, a few updates on use of concomitant medication, e.g., rescue medications, were taken into consideration for efficacy and safety endpoints. Statistically higher percentage of patients receiving lebrikizumab 250 mg vs. placebo achieved IGA (0,1) with ≥2-point improvement from baseline at Week 16 in ADvocate1 (43.1% vs. 12.7% [p<0.001]) and ADvocate2 (33.2% vs. 10.8% [p<0.001]). There was also a higher percentage of patients achieving EASI-75 responses at Week 16 in the lebrikizumab vs. placebo groups in ADvocate1 (58.8% vs. 16.2% [p<0.001]) and ADvocate2 (52.1% vs. 18.1% [p<0.001]); corresponding proportions for EASI-90 at Week 16 were 38.3% vs. 9.0% (p<0.001) in ADvocate1 and 30.7% vs. 9.5% (p<0.001) in ADvocate2. The least-squares mean (LSM) percent change from baseline in the EASI score to Week 16 was significantly greater in lebrikizumab-treated patients (ADvocate1, −64.3; ADvocate2, −61.5; p<0.001) compared with placebo-treated patients (ADvocate1, −26.0; ADvocate2, −28.0; p<0.001). A significantly greater (p<0.001) proportion of patients receiving lebrikizumab 250 mg versus placebo achieved at least 4-point improvement from baseline at Week 16 in Pruritus NRS score (ADvocate1: 45.9% vs. 13% and ADvocate2: 39.8% vs. 11.5%); ≥2-point improvement in Sleep-Loss Scale (ADvocate1: 39% vs. 4.7% and ADvocate2: 28% vs. 8.2%); and ≥4-point improvement in DLQI. In addition, lebrikizumab showed clinically significant improvements in LSM percentage change from baseline in Pruritus NRS score, and LSM change from baseline in Sleep-Loss Scale, and DLQI at week 16 versus the placebo group.

Lebrikizumab 250 mg demonstrated rapid onset of action. In both studies, statistical significance versus placebo was achieved starting at Week 4 for IGA (0,1) with ≥2-point improvement, EASI-90, and Pruritus NRS≥4-point improvement, which were all controlled for multiplicity.

Use of rescue medication was approximately 3-fold and 2-fold greater in placebo-treated patients in ADvocate1 and ADvocate2, respectively, compared with lebrikizumab-treated patients. Patients assigned to placebo treatment required rescue therapy earlier than patients receiving lebrikizumab treatment. The percentage of placebo-treated patients requiring topical and/or systemic rescue therapy as early as Week 2 was 5.0% in ADvocate1 (vs. 1.4% lebrikizumab-treated patients) and 10.3% in ADvocate2 (vs. 3.9% lebrikizumab-treated patients). Rescued patients primarily received topical treatment as opposed to systemic therapy.

Treatment-emergent adverse events (TEAEs) were reported in 51.8% (N=73) and 66.2% (N=96) of placebo patients, compared with 45.7% (N=129) and 53.4% (N=150) of patients receiving lebrikizumab 250 mg, in ADvocate1 and ADvocate2 respectively. Most TEAEs were mild to moderate in severity and resulted in a low frequency of treatment discontinuation. In both studies, low frequency of injection site reactions (ADvocate1, 1.1%; ADvocate2, 2.1%), serious adverse events (ADvocate1, 2.1%; ADvocate2, 0.7%), and TEAEs leading to study discontinuation (ADvocate1, 1.1%; ADvocate2, 3.2%) were reported for patients treated with lebrikizumab 250 mg, comparable to the proportions of patients in the placebo group. One death occurred in the placebo group in ADvocate2. The most common TEAEs (≥5% occurrence in lebrikizumab group and consistently reported with higher frequency than placebo group) was conjunctivitis in ADvocate1 (7.4%) and ADvocate2 (7.5%). All the conjunctivitis-related TEAEs were mild-to-moderate in severity.

Improvements in anxiety and depression were measured in ADvocate 1 and ADvocate 2 using the Patient-Reported Outcomes Measurements Information Systems (PROMIS) scales for anxiety and depression in adults. Missing data were imputed by last observation carried forward (LOCF). In ADvocate1, baseline anxiety scores were 52.9 and 54.3 for patients receiving lebrikizumab 250 mg (N=246) or placebo (N=123); baseline depression scores were 49.8 and 50.0, respectively. Change from baseline (CFB) in anxiety at Week16 was-3.99 for lebrikizumab 250 mg group versus −0.62 for placebo group (p<0.001); depression CFB was −3.16 versus −0.40 (p=0.002), respectively. In ADvocate2, baseline anxiety scores were 54.4 and 55.0, and depression scores were 51.3 and 51.2 for lebrikizumab 250 mg group (N=251) and placebo (N=129) groups, respectively. Anxiety CFB at Week16 was −3.00 for lebrikizumab 250 mg group versus −0.43 for placebo group (p<0.001); depression CFB was −2.38 versus 0.19 (p=0.13), respectively.

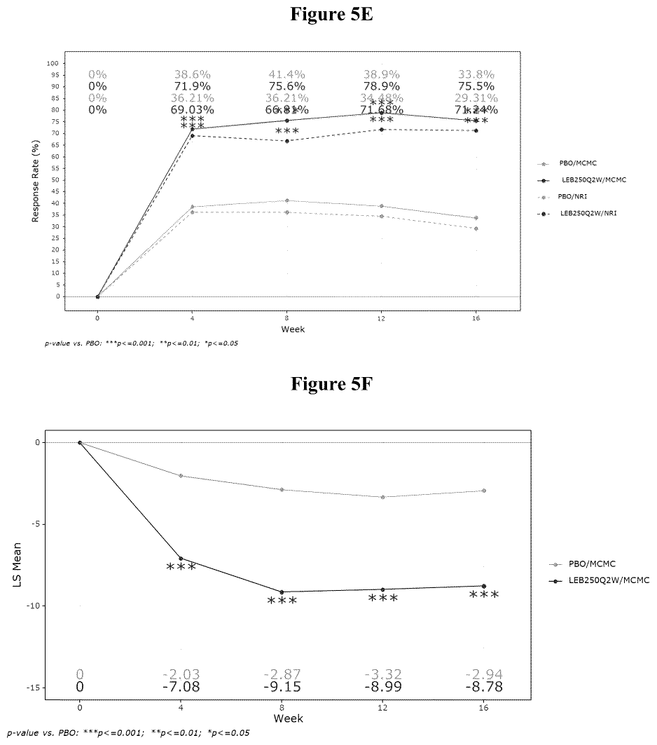

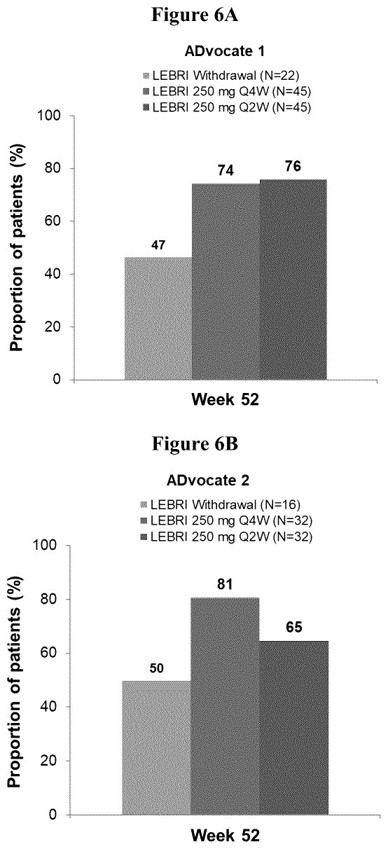

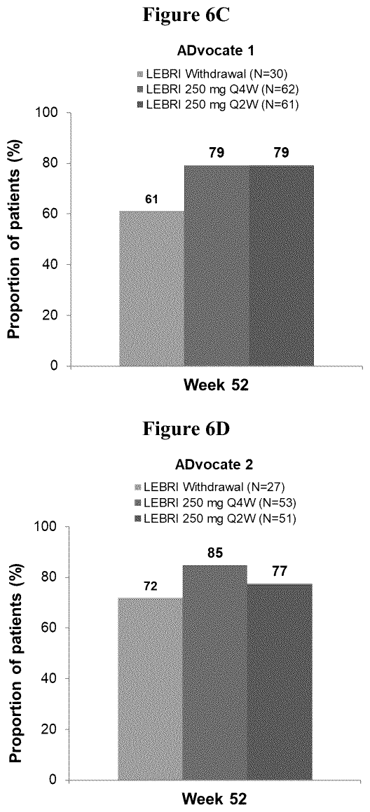

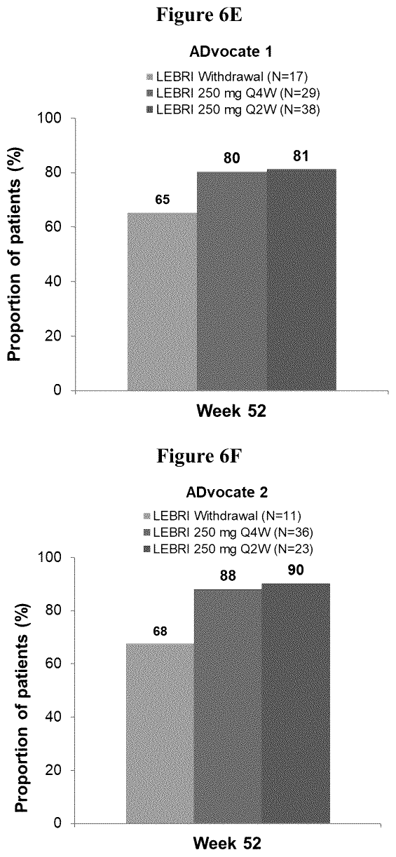

For the maintenance period, in patients who received lebrikizumab treatment during the induction period (or the first period) and are considered responders at Week 16, both lebrikizumab Q4W and Q2W maintenance dosing retained response at Week 52 at a similar rate, surprisingly, as measured by IGA, EASI-75, Pruritus NRS, and in a clinically meaningful percentage of patients (see FIGS. 6A-6F). In ADvocate 1, 79% of patients who received lebrikizumab Q4W and 79% of patients who received lebrikizumab Q2W maintained EASI-75 at Week 52 (see FIG. 6C); 74% of patients who received lebrikizumab Q4W and 76% of patients who received lebrikizumab Q2W maintained IGA of 0 or 1 with a ≥2 point improvement at Week 52. Additionally, in ADvocate 2, 85% of patients who received lebrikizumab Q4W and 77% of patients who received lebrikizumab Q2W maintained EASI-75 response at Week 52 (see FIG. 6D); 81% of patients who received lebrikizumab Q4W and 65% of patients who received lebrikizumab Q2W maintained IGA of 0 or 1 with a ≥2 point improvement at Week 52. In ADvocate 1 and ADvocate 2, 81.2% and 90.3% of patients on lebrikizumab Q2W maintained a ≥4-point improvement from baseline to Week 52 on the Pruritus Numeric Rating Scale (NRS) versus 80.4% and 88.1% of patients on lebrikizumab Q4W, respectively. Overall, about 80% of lebrikizumab responders at Week 16 in ADvocate 1 and 2 maintained improvements in skin clearance and disease severity at Week 52; lasting improvements in itch were also observed at Week 52 in patients treated with lebrikizumab. Unexpectedly, loss of response following withdrawal of lebrikizumab was relatively slow, with approximately half of patients re-randomized to placebo during the maintenance period (the “lebrikizumab withdraw arm”) still having response at Week 52 (see FIGS. 6A-6F). In ADvocate 1 and ADvocate 2, 61% and 72% of patients in the lebrikizumab withdrawal arm continued to meet EASI-75 at Week 52, respectively. The proportion of patients in the lebrikizumab withdrawal arm who maintained a Pruritus NRS response was 65.4% (ADvocate 1) and 67.6% (ADvocate 2). Across treatment arms, the proportion of patients using any rescue therapy was 14.0% (ADvocate 1) and 16.4% (ADvocate2).

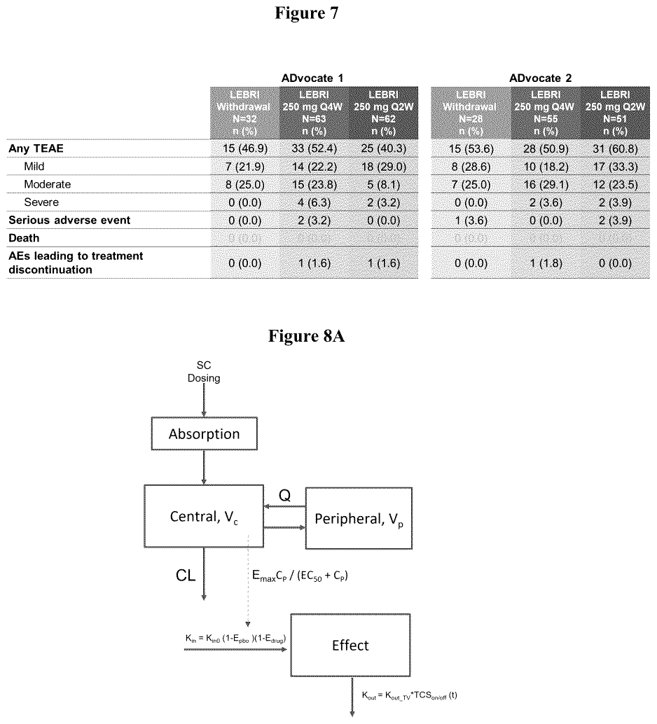

The overall safety profile for lebrikizumab Q2W and Q4W were comparable, with no new safety findings in the maintenance period (see FIG. 7). Low frequency of SAEs and AEs led to discontinuation, no deaths reported. Most reported adverse events were mild to moderate severity. Conjunctivitis for the lebrikizumab treated group across the studies is 14.5%. Only one injection site reaction was reported.

After a 16-week induction with lebrikizumab Q2W, both lebrikizumab Q2W and lebrikizumab Q4W maintained improvement of the signs and symptoms of moderate to severe atopic dermatitis with a good safety profile.

### Example 2. Exposure-Response Modeling and Simulation

The exposure-response relationship for lebrikizumab was evaluated in a combined PK-PD analysis of 5 randomized, double-blind, placebo-controlled studies in patients with AD (Induction Period data (Week 16) from ADvocate 1 and ADvocate 2, NCT04250337, NCT03443024, NCT02340234). The relationship between lebrikizumab exposure and Eczema Area and Severity Index (EASI) response was evaluated based on data from these double-blind placebo-controlled Phase 2 and Phase 3 AD studies. EASI was selected for the modeling since it is a continuous variable. There was a high concordance of 88% between EASI 90 and IGA (0,1) in the lebrikizumab Phase 3 data, thereby further confirming the appropriateness and utility of the EASI endpoint for this type of modeling.

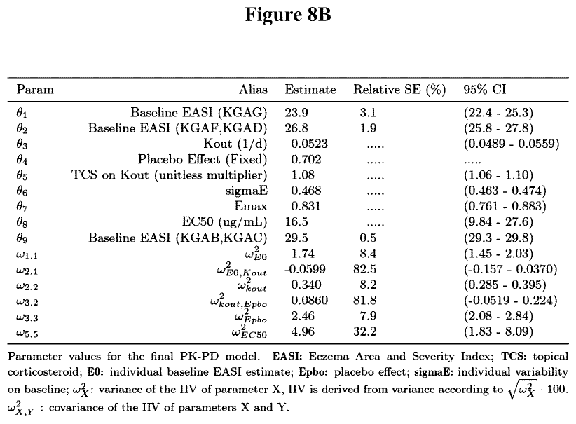

The longitudinal EASI response to lebrikizumab was well described by an indirect-effect model, which included a lebrikizumab drug effect, placebo effect, and time-varying TCS effect. No significant effects of the following covariates were identified in the E-R model: age, sex, body weight (continuous and categorical), race, and baseline IGA (moderate vs severe). The model diagram is shown in FIG. 8A, and the model parameters are shown in FIG. 8B.

After development of the final E-R model, the model was used to explore different aspects of the exposure- and dose-response for lebrikizumab. Simulations using the E-R model were performed to explore maintenance dosing options for participants who had achieved EASI-75 at Week 16 with the induction dosing regimen (500 mg loading doses at Week 0 and 2, followed by 250 mg Q2W to Week 14). Each dosing regimen was simulated for a group of n=125 patients, and the simulations were replicated 500 times. The n=125 was selected based on the typical sample size per treatment arm in Phase 3 studies. In the simulations, the patients who met EASI-75 response at Week 16 were separated into either a responder or nonresponder category, and the data for responders and nonresponders were summarized separately for the various dosing regimens.

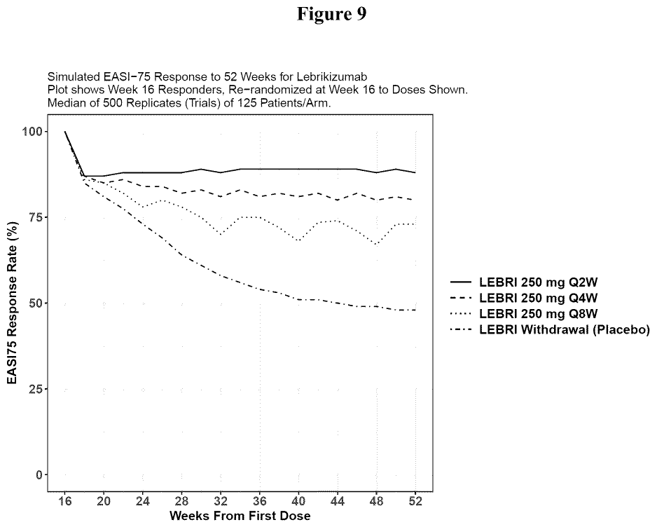

Based on the simulations, a high level of EASI response was predicted for the responders at Week 16 who went to 250 mg Q2W (once every two weeks) or 250 mg Q4W (once every four weeks) maintenance regimens for Week 16 to 52. The EASI-75 and EASI-90 simulations are shown in FIGS. 9 through 12. These simulations were consistent with the observed data from Phase 3 (comparison data not shown).

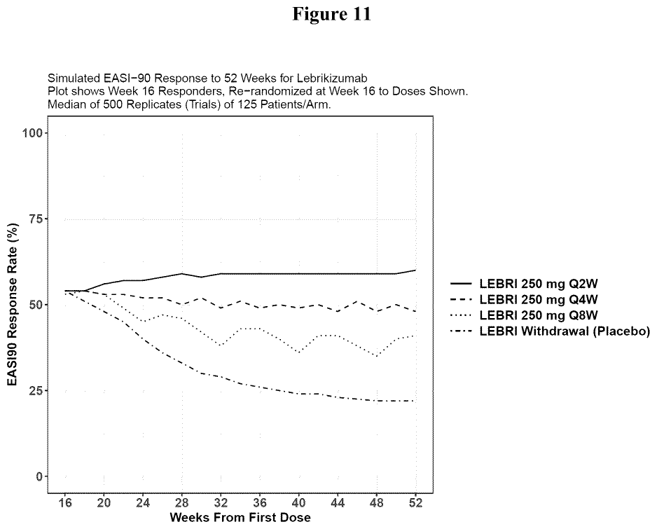

Based on the simulations, a long-lasting EASI response was predicted for responders who went to placebo at Week 16 (FIGS. 9 and 11). The observed efficacy data for the placebo (lebrikizumab withdrawal group) were consistent with simulations using the E-R model (data not shown). The long-lasting effect is attributed to the long PK half-life of lebrikizumab and the indirect E-R relationship.

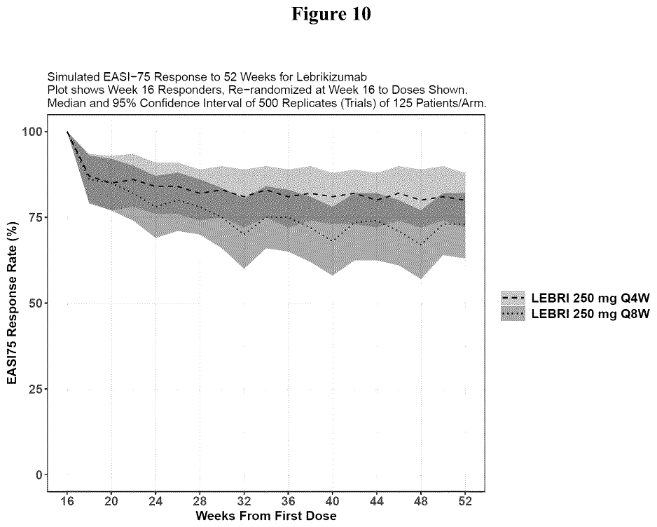

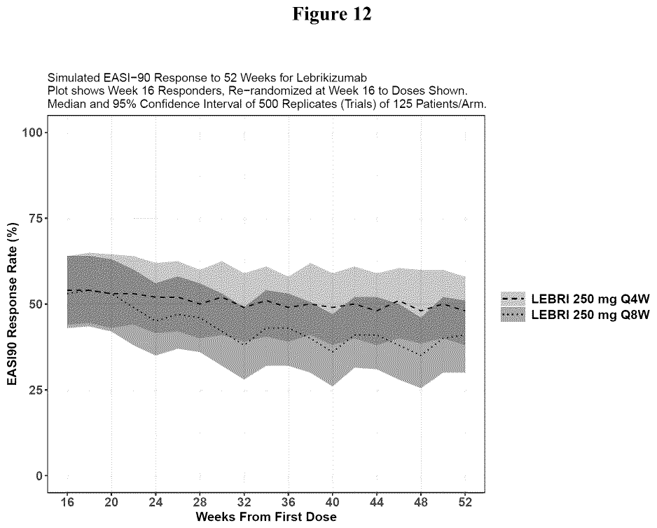

Additional dosing regimen options that were not studied in Phase 3 were explored, including a 250 mg Q8W (once every eight) maintenance regimen after Week 16. A high level of efficacy was predicted for the responders at Week 16 who continued with the 250 mg Q8W maintenance regimen. The EASI-75 and EASI-90 simulations are shown in FIGS. 9-12. The simulations for EASI-75 and EASI-90 are slightly reduced for Q8W dosing compared to Q4W, but Q8W still shows a high level of response (FIGS. 9-12). There was some overlap of the 95% confidence intervals between Q4W and Q8W (FIGS. 10 and 12), also suggesting these two dosing regimens will both result in high levels of efficacy and be comparable to each other.

1. A method of treating moderate to severe atopic dermatitis in a patient in need thereof, the method comprising:
administering to the patient an anti-IL-13 antibody for an induction period of 16 weeks, wherein, during the induction period, the anti-IL-13 antibody is administered at 500 mg at baseline (week 0) and week 2, followed by 250 mg once every two weeks for 14 weeks; and
administering to the patient the anti-IL-13 antibody 250 mg once every four weeks for a maintenance period of 8 to 36 weeks,
wherein the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

2. (canceled)

3. A method of treating moderate to severe atopic dermatitis in a patient in need thereof, the method comprising:
administering to the patient an anti-IL-13 antibody for an induction period of 16 weeks, wherein, during the induction period, the anti-IL-13 antibody is administered at 500 mg at baseline (week 0) and week 2, followed by 250 mg once every two weeks for 14 weeks;
determining if the patient is a responder to the anti-IL-13 antibody after the induction period; and
if the patient is a responder, administering to the patient the anti-IL-13 antibody at 250 mg once every four weeks for a maintenance period of 8 to 36 weeks;
if the patient is not a responder, administering to the patient the anti-IL-13 antibody at 250 mg once every two weeks for a maintenance period of 8 to 36 weeks,
wherein the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

4.-6. (canceled)

7. A method of treating moderate to severe atopic dermatitis in a patient in need thereof, the method comprising:
administering to the patient an anti-IL-13 antibody for an induction period of 16 weeks, wherein, during the induction period, the anti-IL-13 antibody is administered at 500 mg at baseline (week 0) and week 2, followed by 250 mg once every two weeks for 14 weeks; and
administering to the patient the anti-IL-13 antibody 250 mg once every eight weeks for a maintenance period of 8 to 36 weeks,
wherein the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

8. A method of treating moderate to severe atopic dermatitis in a patient in need thereof, the method comprising:
administering to the patient an anti-IL-13 antibody for an induction period of 16 weeks, wherein, during the induction period, the anti-IL-13 antibody is administered at 500 mg at baseline (week 0) and week 2, followed by 250 mg once every two weeks for 14 weeks;
determining if the patient is a responder to the anti-IL-13 antibody after the induction period; and
if the patient is a responder, administering to the patient the anti-IL-13 antibody at 250 mg once every eight weeks for a maintenance period of 8 to 36 weeks;
if the patient is not a responder, administering to the patient the anti-IL-13 antibody at 250 mg once every two weeks for a maintenance period of 8 to 36 weeks,
wherein the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

9.-10. (canceled)

11. The method of claim 1, wherein the patient has moderate to severe atopic dermatitis for at least a year at the baseline.

12. The method of claim 1, wherein the moderate to severe atopic dermatitis is determined by the American Academy of Dermatology Consensus Criteria for Chronic Atopic Dermatitis.

13. The method of claim 1, wherein, at the baseline, the patient has an EASI score of 16 or greater, an IGA score of 3 or greater, and more than 10% of body surface area (BSA) affected by atopic dermatitis.

14. The method of claim 1, wherein the patient has inadequate response to topical corticosteroids, topical calcineurin inhibitors, or crisaborole; or topical corticosteroids, topical calcineurin inhibitors, or crisaborole are medically inadvisable for the patient.

15. The method of claim 1, wherein the patient is aged 12 years and older.

16. The method of claim 1, further comprising determining the patient's EASI score at baseline and during and after the induction period.

17. The method of claim 1, further comprising determining the patient's IGA score at baseline and during and after the induction period.

18. The method of claim 1, further comprising determining one or more of the following characteristics of the patient at baseline and during and after the induction period: the percentage of BSA affected by atopic dermatitis; pruritus NRS score; SCORAD score; sleep loss score; POEM total score; DLQI or CDLQI score; EQ-5D; ACQ-5; PROMIS Anxiety and Depressive Symptoms.

19. The method of claim 3, wherein the patient is a responder when the patient's EASI score determined after the induction period is reduced by 75% or greater compared to the patient's EASI score at the baseline.

20. The method of claim 3, wherein the patient is a responder when the patient's IGA score is 0 or 1 after the induction period, and the patient's IGA score determined after the induction period is reduced by 2 points or greater compared to the patient's IGA score at the baseline.

21.-23. (canceled)

24. The method of claim 1, wherein the maintenance period is 36 weeks.

25.-36. (canceled)

37. The method of claim 1, wherein the antibody comprises a VH comprising SEQ ID NO: 7, and a VL comprising SEQ ID NO: 8.

38. The method of claim 1, wherein the antibody comprises a heavy chain comprising SEQ ID NO: 9, and a light chain comprising SEQ ID NO: 10.

39. The method of claim 1, wherein the antibody is lebrikizumab.

40. The method of claim 1, wherein the anti-IL-13 antibody is administered to the patient subcutaneously.

41. The method of claim 1, wherein the anti-IL-13 antibody is administered to the patient using a subcutaneous administration device.

42. The method of claim 41, wherein the subcutaneous administration device is selected from a prefilled syringe, disposable pen injection device, microneedle device, microinfuser device, needle-free injection device, or autoinjector device.

43. The method of claim 1, wherein the method further comprises administrating one or more topical corticosteroids to the patient.

44. The method of claim 43, wherein the one or more topical corticosteroids is triamcinolone acetonide, hydrocortisone, or a combination of triamcinolone acetonide and hydrocortisone.

45. The method of claim 43, wherein the one or more topical corticosteroids is administered concomitantly with the antibody.

46.-49. (canceled)


SEQUENCE LISTING (10 sequences)

SEQ ID NO: 1 - protein, 5 aa, synthetic construct
  REGION 1..5: note = Synthetic Construct
AYSVN

SEQ ID NO: 2 - protein, 16 aa, synthetic construct
  REGION 1..16: note = Synthetic Construct
MIWGDGKIVYNSALKS

SEQ ID NO: 3 - protein, 10 aa, synthetic construct
  REGION 1..10: note = Synthetic Construct
DGYYPYAMDN

SEQ ID NO: 4 - protein, 15 aa, synthetic construct
  REGION 1..15: note = Synthetic Construct
RASKSVDSYGNSFMH

SEQ ID NO: 5 - protein, 7 aa, synthetic construct
  REGION 1..7: note = Synthetic Construct
LASNLES

SEQ ID NO: 6 - protein, 9 aa, synthetic construct
  REGION 1..9: note = Synthetic Construct
QQNNEDPRT

SEQ ID NO: 7 - protein, 117 aa, synthetic construct
  REGION 1..117: note = Synthetic Construct
VTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 8 - protein, 111 aa, synthetic construct [sequence omitted]

SEQ ID NO: 9 - protein, 445 aa, synthetic construct
  REGION 1..445: note = Synthetic Construct
  SITE 1: note = MISC_FEATURE - X at position 1 is Q, pE, or absent
  SITE 444: note = MISC_FEATURE - X at position 444 is G or absent
  SITE 445: note = MISC_FEATURE - X at position 445 is K or absent
XVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLXX

SEQ ID NO: 10 - protein, 218 aa, synthetic construct
  REGION 1..218: note = Synthetic Construct
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC